(19) United States
(12) Patent Application Publication
(10) Pub. No.: US 2023/0221330 A1
(43) Pub. Date: Jul. 13, 2023

(54) LABELED BINDING REAGENTS AND METHODS OF USE THEREOF

(71) Applicant: Quantum-Si Incorporated, Guilford, CT (US)

(72) Inventors: Brian Reed, Madison, CT (US); Todd Rearick, Cheshire, CT (US); Gerard Schmid, Guilford, CT (US)

(73) Assignee: Quantum-Si Incorporated, Guilford, CT (US)

(21) Appl. No.: 18/153,093

(22) Filed: Jan. 11, 2023

Related U.S. Application Data
(60) Provisional application No. 63/298,972, filed on Jan. 12, 2022.

(51) Int. Cl. G01N 33/68; G01N 33/542; G01N 33/53

(52) U.S. Cl. CPC G01N 33/6818; G01N 33/542; G01N 33/5308; G01N 2333/948

(57) ABSTRACT

Aspects of the disclosure provide methods of identifying and sequencing proteins, polypeptides, and amino acids, and compositions useful for the same. In some aspects, the disclosure provides amino acid recognition molecule compositions, such as amino acid binding proteins comprising different labels, and methods of polypeptide sequencing using such compositions.

### RELATED APPLICATIONS

This application claims the benefit under 35 U.S.C. § 119(e) of U.S. Provisional Application Ser. No. 63/298,972, filed Jan. 12, 2022, under Attorney Docket No.: R0708.70147US00, and entitled, “LABELED BINDING REAGENTS AND METHODS OF USE THEREOF,” which is herein incorporated by reference in its entirety.

### REFERENCE TO AN ELECTRONIC SEQUENCE LISTING

The contents of the electronic sequence listing (R070870147US01-SEQ-RJP.xml; Size: 8,341 bytes; and Date of Creation: Jan. 11, 2023) is herein incorporated by reference in its entirety.

### BACKGROUND

Proteomics has emerged as an important and necessary complement to genomics and transcriptomics in the study of biological systems. The proteomic analysis of an individual organism can provide insights into cellular processes and response patterns, which lead to improved diagnostic and therapeutic strategies. The complexity surrounding protein structure, composition, and modification present challenges in determining large-scale protein sequencing information for a biological sample.

### SUMMARY

In some aspects, the disclosure provides methods and compositions for determining amino acid sequence information from polypeptides. In some embodiments, amino acid sequence information can be determined by contacting a single polypeptide molecule with one or more amino acid recognition molecules comprising uniquely identifiable detectable labels, where each detectable label is associated with a type of amino acid (or subset of types) to which the amino acid recognition molecule binds.

In some embodiments, an amino acid recognition molecule of the disclosure comprises a detectable label that undergoes Forster/fluorescence resonance energy transfer (FRET). In some embodiments, amino acid sequence information can be determined by contacting a single polypeptide molecule with at least two amino acid binding proteins comprising different FRET labels. In some embodiments, the different FRET labels comprise different configurations of chromophores of the same type. In some embodiments, the different configurations permit different FRET efficiencies, such that the different FRET labels (and the different types of amino acids associated therewith) may be distinguishable by relative emission intensities of donor and acceptor chromophores.

In some embodiments, the disclosure provides compositions comprising two or more types of amino acid recognition molecules, where each type binds the same type of amino acid and comprises a different type of label. For example, in some embodiments, the composition comprises a first and second amino acid binding protein comprising a first and second label, respectively, where the first label is different from the second label, and where the first and second amino acid binding proteins binds the same type of amino acid. Such compositions can be used in polypeptide sequencing reactions to provide increased confidence levels in determining the identity of an amino acid of a polypeptide.

In some aspects, the disclosure provides a composition comprising: a first amino acid binding protein comprising a first FRET label, where the first FRET label has a first emission spectrum comprising peaks of a first wavelength and a second wavelength; and a second amino acid binding protein comprising a second FRET label, where the second FRET label has a second emission spectrum comprising peaks of the first wavelength and the second wavelength.

In some embodiments, emission intensities at one or both peaks of the first emission spectrum are different from emission intensities at one or both peaks of the second emission spectrum. In some embodiments, each peak is characterized by an emission intensity at a particular wavelength (e.g., the first or second wavelength), and the emission intensity at the particular wavelength in the first and second emission spectra are different. In some embodiments, emission intensities at the first and second wavelengths in the first emission spectrum are different from emission intensities at the first and second wavelengths in the second emission spectrum. For example, in some embodiments, emission intensity at the first wavelength in the first emission spectrum is different from emission intensity at the first wavelength in the second emission spectrum, and emission intensity at the second wavelength in the first emission spectrum is different from emission intensity at the second wavelength in the second emission spectrum.

In some embodiments, the first wavelength is an emission wavelength for a donor chromophore of each FRET label, and the second wavelength is an emission wavelength for an acceptor chromophore of each FRET label. In some embodiments, the ratio of the donor chromophore to the acceptor chromophore in each FRET label is 1:1, 2:1, 3:1, 4:1, 5:1, 1:2, 1:3, 1:4, or 1:5.

In some embodiments, the first FRET label has a first FRET efficiency, and the second FRET label has a second FRET efficiency, where the first FRET efficiency is different from the second FRET efficiency. In some embodiments, the first FRET efficiency differs from the second FRET efficiency by at least about 5%. In some embodiments, the first amino acid binding protein comprises the first FRET label in a first configuration that permits the first FRET efficiency; and the second amino acid binding protein comprises the second FRET label in a second configuration that permits the second FRET efficiency. In some embodiments, the first configuration maintains a first distance between chromophores in the first FRET label, and the second configuration maintains a second distance between the chromophores in the second FRET label, where the first distance is different from the second distance.

In some embodiments, the first amino acid binding protein is attached to the first FRET label through a first linkage group, and the second amino acid binding protein is attached to the second FRET label through a second linkage group. In some embodiments, chromophores of the first FRET label are attached to the first linkage group in the first configuration, and chromophores of the second FRET label are attached to the second linkage group in the second configuration.

In some embodiments, the first FRET label comprises a first chromophore, and the second FRET label comprises a second chromophore that is identical to the first chromophore. In some embodiments, the first FRET label comprises a first plurality of chromophores, and the second FRET label comprises a second plurality of chromophores, where chromophores of the first plurality are identical to chromophores of the second plurality.

In some embodiments, the composition further comprises at least one amino acid binding protein comprising a non-FRET label. In some embodiments, the non-FRET label comprises a fluorophore. In some embodiments, the non-FRET label comprises a chromophore identical to a donor or acceptor chromophore of the first FRET label.

In some embodiments, the first emission spectrum distinctly identifies a first type of amino acid, and the second emission spectrum distinctly identifies a second type of amino acid. In some embodiments, the first and second types of amino acids are naturally occurring amino acids of a different type. In some embodiments, the first amino acid binding protein binds to a first subset of types of amino acids, and the second amino acid binding protein binds to a second subset of types of amino acids. In some embodiments, the first subset of types of amino acids is different from the second subset of types of amino acids.

In some embodiments, the composition further comprises at least one peptidase. In some embodiments, the molar ratio of the first or second amino acid binding protein to the peptidase is between about 1:1,000 and about 1:1 or between about 1:1 and about 100:1. In some embodiments, the molar ratio of the first or second amino acid binding protein to the peptidase is between about 1:100 and about 1:1 or between about 1:1 and about 10:1. In some embodiments, the molar ratio of the first or second amino acid binding protein to the peptidase is about 1:1,000, about 1:500, about 1:200, about 1:100, about 1:10, about 1:5, about 1:2, about 1:1, about 5:1, about 10:1, about 50:1, about 100:1.

In some embodiments, the first and second amino acid binding proteins are each independently selected from a Gid protein, a UBR-box protein or UBR-box domain-containing fragment thereof, a p62 protein or ZZ domain-containing fragment thereof, and a ClpS protein. In some embodiments, at least one of the first and second amino acid binding proteins is a ClpS protein.

In some aspects, the disclosure provides a labeled amino acid recognition molecule comprising: a nucleic acid comprising a FRET label, where the FRET label has an emission spectrum comprising at least two peaks that distinctly identify a terminal amino acid; and at least one amino acid binding protein attached to the nucleic acid, where the nucleic acid forms a covalent or non-covalent linkage group between the at least one amino acid binding protein and the FRET label.

In some embodiments, the FRET label has a FRET efficiency of less than 90%. In some embodiments, the FRET label is attached to the nucleic acid in a configuration that permits the FRET efficiency. In some embodiments, the FRET label comprises a plurality of chromophores attached to a respective plurality of attachment sites on the nucleic acid. In some embodiments, each attachment site is separated by another attachment site of the plurality by between 5 and 100 nucleotide bases or nucleotide base pairs on the nucleic acid.

In some embodiments, the FRET label is attached to the nucleic acid through a biomolecule that forms a covalent or non-covalent linkage group between the FRET label and the nucleic acid. In some embodiments, the FRET label comprises a plurality of chromophores attached to a respective plurality of attachment sites on the biomolecule. In some embodiments, the biomolecule is a multivalent protein.

In some embodiments, the nucleic acid is a double-stranded nucleic acid comprising a first oligonucleotide strand hybridized with a second oligonucleotide strand. In some embodiments, the at least one amino acid binding protein is attached to the first oligonucleotide strand, where the FRET label is attached to the first oligonucleotide strand. In some embodiments, the at least one amino acid binding protein is attached to the first oligonucleotide strand, and where the FRET label is attached to the second oligonucleotide strand. In some embodiments, the at least one amino acid binding protein is attached to the first oligonucleotide strand, where chromophores of the FRET label are attached to each of the first and second oligonucleotide strands.

In some embodiments, the FRET label comprises a donor chromophore and an acceptor chromophore, where the ratio of the donor chromophore to the acceptor chromophore is 1:1, 2:1, 3:1, 4:1, 5:1, 1:2, 1:3, 1:4, or 1:5.

In some aspects, the disclosure provides a composition comprising: a first amino acid binding protein comprising a first label, where the first amino acid binding protein binds a first type of amino acid; and a second amino acid binding protein comprising a second label, where the second amino acid binding protein binds the first type of amino acid, and where the first label is different from the second label.

In some embodiments, the first and second amino acid binding proteins are the same. In some embodiments, the first and second amino acid binding proteins are different. In some embodiments, the first amino acid binding protein binds the first type of amino acid with a first dissociation rate, and the second amino acid binding protein binds the first type of amino acid with a second dissociation rate, where the first dissociation rate is different from the second dissociation rate.

In some embodiments, the first label comprises a first fluorophore, and the second label comprises a second fluorophore, where the first fluorophore is different from the second fluorophore. In some embodiments, the first and second amino acid binding proteins are each independently selected from a Gid protein, a UBR-box protein or UBR-box domain-containing fragment thereof, a p62 protein or ZZ domain-containing fragment thereof, and a ClpS protein. In some embodiments, at least one of the first and second amino acid binding proteins is a ClpS protein.

In some aspects, the disclosure provides methods of polypeptide sequencing, the methods comprising: contacting a single polypeptide molecule with a composition described herein which comprises at least a first amino acid binding protein and a second amino acid binding protein; and detecting a series of signal pulses indicative of association of the first and second amino acid binding proteins with the single polypeptide while the single polypeptide is being degraded, thereby sequencing the single polypeptide molecule.

In some aspects, the disclosure provides methods of identifying a terminal amino acid of a polypeptide, the methods comprising: contacting a single polypeptide molecule with a composition described herein which comprises at least a first amino acid binding protein and a second amino acid binding protein; and detecting a series of signal pulses indicative of association of the first and second amino acid binding proteins with a terminus of the single polypeptide molecule; and identifying the first type of amino acid at the terminus of the single polypeptide molecule based on a characteristic pattern in the series of signal pulses.

In some embodiments, a signal pulse of the characteristic pattern corresponds to an individual association event between the first or second amino acid binding protein and the first type of amino acid. In some embodiments, the signal pulse of the characteristic pattern comprises a pulse duration that is characteristic of a dissociation rate of binding between the first or second amino acid binding protein and the first type of amino acid. In some embodiments, association of the first amino acid binding protein with the first type of amino acid produces a first pulse duration, and association of the second amino acid binding protein with the first type of amino acid produces a second pulse duration. In some embodiments, the first pulse duration is different from the second pulse duration. In some embodiments, the first and second pulse durations are the same.

In some aspects, the disclosure provides an integrated device comprising: at least one chamber for receiving one or more labeled amino acid binding proteins; at least one photodetection region for receiving a signal emitted by the one or more labeled amino acid binding proteins in response to excitation light from at least one light source, the signal including information representative of at least one characteristic of the one or more labeled amino acid binding proteins; and at least one controller configured to obtain one or more adjusted measurements by controlling adjusting of one or more subsequent measurements obtained from a single polypeptide molecule disposed in the at least one chamber based on the information obtained from the signal emitted by the one or more labeled amino acid binding proteins.

In some embodiments, the one or more labeled amino acid binding proteins comprise at least one amino acid binding protein comprising a FRET label, where the FRET label has an emission spectrum comprising peaks of a first wavelength and a second wavelength. In some embodiments, the one or more labeled amino acid binding proteins comprise: a first amino acid binding protein comprising a first FRET label, where the first FRET label has a first emission spectrum comprising peaks of a first wavelength and a second wavelength; and a second amino acid binding protein comprising a second FRET label, where the second FRET label has a second emission spectrum comprising peaks of the first wavelength and the second wavelength, and where emission intensities at the first and second wavelengths in the first emission spectrum are different from emission intensities at the first and second wavelengths in the second emission spectrum.

In some embodiments, the one or more labeled amino acid binding proteins comprise: a first amino acid binding protein comprising a first label, where the first amino acid binding protein binds a first type of amino acid; and a second amino acid binding protein comprising a second label, where the second amino acid binding protein binds the first type of amino acid, and where the first label is different from the second label.

In some embodiments, the at least one characteristic of the labeled amino acid binding protein comprises a luminescence intensity, a luminescence wavelength, a luminescence lifetime, a pulse duration, and/or an interpulse duration. In some embodiments, the one or more adjusted measurements are representative of a luminescence intensity, a luminescence wavelength, a luminescence lifetime, a pulse duration, and/or an interpulse duration.

In some embodiments, the at least one controller is configured to identify one or more amino acids of the single polypeptide molecule based at least in part on the one or more adjusted measurements. In some embodiments, the at least one controller is configured to identify the single polypeptide molecule, or a protein from which the single polypeptide molecule is derived, at least in part by identifying one or more amino acids of the single polypeptide molecule based at least in part on the one or more adjusted measurements.

In some embodiments, the at least one chamber comprises a plurality of chambers having a respective plurality of single polypeptide molecules disposed therein. In some embodiments, the one or more labeled amino acid binding proteins comprise a plurality of labeled amino acid binding proteins. In some embodiments, the at least one photodetection region comprises a plurality of photodetection regions configured to receive signals from the plurality of labeled amino acid binding proteins. In some embodiments, the at least one controller is configured to control the adjusting of the one or more subsequent measurements obtained respectively from each of the plurality of single polypeptide molecules based on information obtained from the plurality of signals emitted by the plurality of labeled amino acid binding proteins.

In some aspects, the disclosure provides methods and compositions for determining amino acid sequence information from polypeptides (e.g., for sequencing one or more polypeptides). In some embodiments, amino acid sequence information can be determined for single polypeptide molecules. In some embodiments, the relative position of two or more amino acids in a polypeptide is determined, for example for a single polypeptide molecule. In some embodiments, amino acid sequence information can be determined by detecting an interaction of a polypeptide with one or more amino acid recognition molecules (e.g., one or more amino acid binding proteins).

In some aspects, the disclosure provides an amino acid binding protein which can be used in a method for determining amino acid sequence information from polypeptides. In some aspects, the disclosure provides a recombinant amino acid binding protein comprising one or more labels. In some embodiments, the one or more labels comprise a luminescent label or a conductivity label. In some embodiments, the one or more labels comprise a FRET label as described herein. In some embodiments, the one or more labels comprise a tag sequence. In some embodiments, the tag sequence comprises one or more of a purification tag, a cleavage site, and a biotinylation sequence (e.g., at least one biotin ligase recognition sequence). In some embodiments, the biotinylation sequence comprises two biotin ligase recognition sequences oriented in tandem. In some embodiments, the one or more labels comprise a biotin moiety having at least one biotin molecule (e.g., a bis-biotin moiety). In some embodiments, the label comprises at least one biotin ligase recognition sequence having the at least one biotin molecule attached thereto. In some embodiments, the one or more labels comprise one or more polyol moieties (e.g., polyethylene glycol). In some embodiments, the recombinant amino acid binding protein comprises one or more unnatural amino acids having the one or more labels attached thereto. In some aspects, the disclosure provides a composition comprising a recombinant amino acid binding protein described herein.

In some aspects, the disclosure provides a polypeptide sequencing reaction composition comprising two or more amino acid recognition molecules, where at least one of the two or more amino acid recognition molecules is a recombinant amino acid binding protein described herein. In some embodiments, the two or more amino acid recognition molecules comprise different types of amino acid recognition molecules. For example, in some embodiments, an amino acid recognition molecule of one type interacts with a polypeptide of interest in a manner that is different (e.g., detectably different) from other types of amino acid recognition molecules in a polypeptide sequencing reaction composition. In some embodiments, the polypeptide sequencing reaction composition comprises at least one type of cleaving reagent. In some aspects, the disclosure provides a method of polypeptide sequencing comprising contacting a polypeptide with a polypeptide sequencing reaction composition described herein. In some embodiments, the method further comprises detecting a series of interactions of the polypeptide with at least one amino acid recognition molecule while the polypeptide is being degraded, thereby sequencing the polypeptide.

In some aspects, the disclosure provides a polypeptide sequencing reaction mixture comprising an amino acid binding protein and a peptidase. In some embodiments, the molar ratio of the labeled amino acid binding protein to the peptidase is between about 1:1,000 and about 1:1 or between about 1:1 and about 100:1. In some embodiments, the amino acid binding protein comprises one or more labels. In some embodiments, the one or more labels comprise a FRET label as described herein. In some embodiments, the amino acid binding protein is a ClpS protein. In some embodiments, the peptidase is an exopeptidase. In some embodiments, the reaction mixture comprises more than one amino acid binding protein and/or more than one peptidase. In some embodiments, the reaction mixture comprises a polypeptide molecule immobilized to a surface.

In some aspects, the disclosure provides a polypeptide sequencing reaction mixture comprising a single polypeptide molecule, at least one peptidase molecule, and at least three amino acid recognition molecules. In some embodiments, the at least three amino acid recognition molecules include at least a first amino acid binding protein comprising a first FRET label and a second amino acid binding protein comprising a second FRET label. In some embodiments, the reaction mixture comprises at least 1 and up to 10 peptidase molecules (e.g., at least 1 and up to 5 peptidase molecules, at least 1 and up to 3 peptidase molecules). In some embodiments, the reaction mixture comprises two or more peptidase molecules, where each peptidase molecule is of a different type. For example, in some embodiments, a peptidase molecule of one type has a cleavage preference that is different from other types of peptidase molecules in a reaction mixture. In some embodiments, the reaction mixture comprises at least 3 and up to 30 amino acid recognition molecules (e.g., up to 20, up to 10, or up to 5 amino acid recognition molecules). In some embodiments, the at least three amino acid recognition molecules comprise different types of amino acid recognition molecules. For example, in some embodiments, an amino acid recognition molecule of one type interacts with a polypeptide of interest in a manner that is different (e.g., detectably different) from other types of amino acid recognition molecules in a reaction mixture.

In some aspects, the disclosure provides a substrate comprising an array of sample wells, where at least one sample well of the array comprises a polypeptide sequencing reaction mixture described herein. In some embodiments, the at least one sample well comprises a bottom surface. In some embodiments, the single polypeptide molecule is immobilized to the bottom surface.

In some aspects, the disclosure provides an amino acid recognition molecule comprising a polypeptide having at least a first amino acid binding protein and a second amino acid binding protein joined end-to-end, where the first and second amino acid binding proteins are separated by a linker comprising at least two amino acids. In some embodiments, the first and second amino acid binding proteins are the same. In some embodiments, the first and second amino acid binding proteins are different. In some embodiments, the amino acid recognition molecule comprises a FRET label as described herein.

In some aspects, the disclosure provides an amino acid recognition molecule comprising a polypeptide of Formula (I):

(Z¹—X¹)ₙ—Z² (I),

wherein: Z¹ and Z² are independently amino acid binding proteins; X¹ is a linker comprising at least two amino acids, where the amino acid binding proteins are joined end-to-end by the linker; and n is an integer from 1 to 5, inclusive. In some embodiments, Z¹ and Z² comprise amino acid binding proteins of the same type. In some embodiments, Z¹ and Z² comprise different types of amino acid binding proteins. In some embodiments, Z¹ and Z² are independently optionally associated with a label component comprising at least one detectable label. In some embodiments, the label component comprises a FRET label as described herein. In some embodiments, the polypeptide further comprises a tag sequence.

In some aspects, the disclosure provides methods of polypeptide sequencing. In some embodiments, a method of polypeptide sequencing comprises contacting a single polypeptide molecule in a reaction mixture with a composition comprising a binding means and a cleaving means. In some embodiments, the binding means and the cleaving means are configured to achieve at least 10 association events between the binding means and a terminal amino acid on the polypeptide prior to removal of the terminal amino acid from the polypeptide by the cleaving means. In some embodiments, the binding means and the cleaving means are configured to achieve at least 10 and up to 1,000 association events prior to the removal of the terminal amino acid. In some embodiments, the terminal amino acid was exposed at the polypeptide terminus in a cleavage event prior to the at least 10 association events. In some embodiments, the at least 10 association events occur after the cleavage event.

In some embodiments, the binding means and the cleaving means are configured to achieve a time interval of at least 1 minute between cleavage events (e.g., between about 1 minute and about 20 minutes, between about 5 minutes and about 15 minutes, or between about 1 minute and about 10 minutes). In some embodiments, the binding means comprise one or more amino acid recognition molecules, and the cleaving means comprise one or more peptidase molecules. In some embodiments, the one or more amino acid recognition molecules include at least a first amino acid binding protein comprising a first FRET label and a second amino acid binding protein comprising a second FRET label. In some embodiments, the molar ratio of an amino acid recognition molecule to a peptidase molecule is configured to achieve the at least 10 association events prior to the removal of the terminal amino acid. In some embodiments, the molar ratio of the amino acid recognition molecule to the peptidase molecule is between about 1:1,000 and about 1:1 or between about 1:1 and about 100:1. In some embodiments, the molar ratio of the amino acid recognition molecule to the peptidase molecule is between about 1:100 and about 1:1 or between about 1:1 and about 10:1.

In some aspects, the disclosure provides a substrate comprising an array of sample wells, where at least one sample well of the array comprises a single polypeptide molecule, a cleaving means, and a binding means. In some embodiments, the binding means and the cleaving means are configured to achieve at least 10 association events between the binding means and a terminal amino acid on the polypeptide prior to removal of the terminal amino acid from the polypeptide by the cleaving means. In some embodiments, the binding means and the cleaving means are configured to achieve at least 10 and up to 1,000 association events prior to the removal of the terminal amino acid. In some embodiments, the terminal amino acid was exposed at the polypeptide terminus in a cleavage event prior to the at least 10 association events. In some embodiments, the at least 10 association events occur after the cleavage event.

In some aspects, the disclosure provides amino acid recognition molecules comprising a shielding element, e.g., for enhanced photostability in polypeptide sequencing reactions. In some aspects, the disclosure provides an amino acid recognition molecule comprising a polypeptide having an amino acid binding protein and a labeled protein joined end-to-end. In some embodiments, the labeled protein is a protein comprising a FRET label as described herein. In some embodiments, the amino acid binding protein and the labeled protein are separated by a linker comprising at least two amino acids (e.g., at least two and up to 100 amino acids, between about 5 and about 50 amino acids). In some embodiments, the labeled protein has a molecular weight of at least 10 kDa (e.g., between about 10 kDa and about 150 kDa, between about 15 kDa and about 100 kDa). In some embodiments, the labeled protein comprises at least 50 amino acids (e.g., between about 50 and about 1,000 amino acids, between about 100 and about 750 amino acids). In some embodiments, the labeled protein comprises a luminescent label. In some embodiments, the luminescent label comprises at least one fluorophore dye molecule. In some embodiments, the amino acid binding protein is a Gid protein, a UBR-box protein or UBR-box domain-containing fragment thereof, a p62 protein or ZZ domain-containing fragment thereof, or a ClpS protein.

In some aspects, the disclosure provides an amino acid recognition molecule of Formula (II):

A-(Y)ₙ-D (II),

wherein: A is an amino acid binding component comprising at least one amino acid recognition molecule; each instance of Y is a polymer that forms a covalent or non-covalent linkage group; n is an integer from 1 to 10, inclusive; and D is a label component comprising at least one detectable label. In some embodiments, A comprises at least one amino acid binding protein. In some embodiments, the amino acid recognition molecule comprises a polypeptide having A and Y¹ joined end-to-end, wherein A and Y¹ are separated by a linker comprising at least two amino acids. In some embodiments, Y¹ is a protein having a molecular weight of at least 10 kDa (e.g., between about 10 kDa and about 150 kDa). In some embodiments, Y¹ is a protein comprising at least 50 amino acids (e.g., between about 50 and about 1,000 amino acids).

In some embodiments, D is a FRET label as described herein. In some embodiments, D is less than 200 A in diameter. In some embodiments, —(Y)ₙ— is at least 2 nm in length (e.g., at least 5 nm, at least 10 nm, at least 20 nm, at least 30 nm, at least 50 nm, or more, in length). In some embodiments, —(Y)ₙ— is between about 2 nm and about 200 nm in length (e.g., between about 2 nm and about 100 nm, between about 5 nm and about 50 nm, or between about 10 nm and about 100 nm in length). In some embodiments, each instance of Y is independently a biomolecule or a dendritic polymer (e.g., a polyol, a dendrimer). In some embodiments, A comprises a polypeptide having at least a first amino acid binding protein and a second amino acid binding protein joined end-to-end (e.g., a fusion polypeptide). In some embodiments, the disclosure provides a composition comprising the amino acid recognition molecule of Formula (II). In some embodiments, the amino acid recognition molecule is soluble in the composition.

In some aspects, the disclosure provides an amino acid recognition molecule of Formula (III):

A-Y¹-D (III),

wherein: A is an amino acid binding component comprising at least one amino acid recognition molecule; Y¹ is a nucleic acid or a polypeptide; D is a label component comprising at least one detectable label. In some embodiments, A comprises at least one amino acid binding protein. In some embodiments, when Y¹ is a nucleic acid, the nucleic acid forms a covalent or non-covalent linkage group. In some embodiments, provided that when Y¹ is a polypeptide, the polypeptide forms a non-covalent linkage group characterized by a dissociation constant (K_{D}) of less than 50×10⁻⁹ M. In some embodiments, the K_{D} is less than 1×10⁻⁹ M, less than 1×10⁻¹⁰ M, less than 1×10⁻¹¹ M, or less than 1×10⁻¹² M. In some embodiments, D is a FRET label as described herein.

In some aspects, the disclosure provides an amino acid recognition molecule comprising: a nucleic acid; at least one amino acid recognition molecule attached to a first attachment site on the nucleic acid; and at least one detectable label attached to a second attachment site on the nucleic acid, where the nucleic acid forms a covalent or non-covalent linkage group between the at least one amino acid recognition molecule and the at least one detectable label. In some embodiments, the nucleic acid comprises a first oligonucleotide strand. In some embodiments, the nucleic acid further comprises a second oligonucleotide strand hybridized with the first oligonucleotide strand. In some embodiments, the at least one amino acid recognition molecule comprises a polypeptide having at least a first amino acid binding protein and a second amino acid binding protein joined end-to-end (e.g., a fusion polypeptide). In some embodiments, the first and second amino acid binding proteins are separated by a linker comprising at least two amino acids. In some embodiments, the at least one detectable label comprises a FRET label as described herein.

In some aspects, the disclosure provides an amino acid recognition molecule comprising: a multivalent protein comprising at least two ligand-binding sites; at least one amino acid recognition molecule attached to the protein through a first ligand moiety bound to a first ligand-binding site on the protein; and at least one detectable label attached to the protein through a second ligand moiety bound to a second ligand-binding site on the protein. In some embodiments, the multivalent protein is an avidin protein. In some embodiments, the at least one amino acid recognition molecule comprises a polypeptide having at least a first amino acid binding protein and a second amino acid binding protein joined end-to-end (e.g., a fusion polypeptide). In some embodiments, the first and second amino acid binding proteins are separated by a linker comprising at least two amino acids. In some embodiments, the at least one detectable label comprises a FRET label as described herein.

In some embodiments, a shielded amino acid recognition molecule may be used in polypeptide sequencing methods in accordance with the disclosure, or any method known in the art. Accordingly, in some aspects, the disclosure provides methods of polypeptide sequencing (e.g., in an Edman-type degradation reaction, in a dynamic sequencing reaction, or other method known in the art) comprising contacting a polypeptide molecule with one or more shielded amino acid recognition molecules of the disclosure. For example, in some embodiments, the methods comprise contacting a polypeptide molecule with at least one amino acid recognition molecule that comprises a shield or shielding element in accordance with the disclosure, and detecting association of the at least one amino acid recognition molecule with the polypeptide molecule.

In some aspects, the disclosure provides methods of polypeptide sequencing comprising contacting a single polypeptide molecule with one or more amino acid recognition molecules (e.g., one or more terminal amino acid recognition molecules). In some embodiments, the one or more amino acid recognition molecules include at least a first amino acid binding protein comprising a first FRET label and a second amino acid binding protein comprising a second FRET label. In some embodiments, the methods further comprise detecting a series of signal pulses indicative of association of the one or more amino acid recognition molecules with successive amino acids exposed at a terminus of the single polypeptide molecule while it is being degraded, thereby obtaining sequence information about the single polypeptide molecule. In some embodiments, the amino acid sequence of most or all of the single polypeptide molecule is determined. In some embodiments, the series of signal pulses is a series of real-time signal pulses.

In some embodiments, association of the one or more amino acid recognition molecules with each type of amino acid exposed at the terminus produces a characteristic pattern in the series of signal pulses that is different from other types of amino acids exposed at the terminus. In some embodiments, signal pulses of the characteristic pattern comprise a mean pulse duration of between about 1 millisecond and about 10 seconds. In some embodiments, a signal pulse of the characteristic pattern corresponds to an individual association event between an amino acid recognition molecule and an amino acid exposed at the terminus. In some embodiments, the characteristic pattern corresponds to a series of reversible amino acid recognition molecule binding interactions with the amino acid exposed at the terminus of the single polypeptide molecule. In some embodiments, the characteristic pattern is indicative of the amino acid exposed at the terminus of the single polypeptide molecule and an amino acid at a contiguous position (e.g., amino acids of the same type or different types).

In some embodiments, the single polypeptide molecule is degraded by a cleaving reagent that removes one or more amino acids from the terminus of the single polypeptide molecule. In some embodiments, the methods further comprise detecting a signal indicative of association of the cleaving reagent with the terminus. In some embodiments, the cleaving reagent comprises a detectable label (e.g., a luminescent label, a conductivity label). In some embodiments, the cleaving reagent comprises a FRET label as described herein. In some embodiments, the single polypeptide molecule is immobilized to a surface. In some embodiments, the single polypeptide molecule is immobilized to the surface through a terminal end distal to the terminus to which the one or more amino acid recognition molecules associate. In some embodiments, the single polypeptide molecule is immobilized to the surface through a linker (e.g., a solubilizing linker comprising a biomolecule).

In some aspects, the disclosure provides methods of sequencing a polypeptide comprising contacting a single polypeptide molecule in a reaction mixture with a composition comprising one or more amino acid recognition molecules (e.g., one or more terminal amino acid recognition molecules) and a cleaving reagent. In some embodiments, the one or more amino acid recognition molecules include at least a first amino acid binding protein comprising a first FRET label and a second amino acid binding protein comprising a second FRET label. In some embodiments, the methods further comprise detecting a series of signal pulses indicative of association of the one or more amino acid recognition molecules with a terminus of the single polypeptide molecule in the presence of the cleaving reagent. In some embodiments, the series of signal pulses is indicative of a series of amino acids exposed at the terminus over time as a result of terminal amino acid cleavage by the cleaving reagent.

In some aspects, the disclosure provides methods of sequencing a polypeptide comprising (a) identifying a first amino acid at a terminus of a single polypeptide molecule, (b) removing the first amino acid to expose a second amino acid at the terminus of the single polypeptide molecule, and (c) identifying the second amino acid at the terminus of the single polypeptide molecule. In some embodiments, (a)-(c) are performed in a single reaction mixture. In some embodiments, (a)-(c) occur sequentially. In some embodiments, (c) occurs before (a) and (b). In some embodiments, the single reaction mixture comprises one or more amino acid recognition molecules (e.g., one or more terminal amino acid recognition molecules). In some embodiments, the one or more amino acid recognition molecules include at least a first amino acid binding protein comprising a first FRET label and a second amino acid binding protein comprising a second FRET label. In some embodiments, the single reaction mixture comprises a cleaving reagent. In some embodiments, the first amino acid is removed by the cleaving reagent. In some embodiments, the methods further comprise repeating the steps of removing and identifying one or more amino acids at the terminus of the single polypeptide molecule, thereby determining a sequence (e.g., a partial sequence or a complete sequence) of the single polypeptide molecule.

In some aspects, the disclosure provides methods of identifying an amino acid of a polypeptide comprising contacting a single polypeptide molecule with one or more amino acid recognition molecules that bind to the single polypeptide molecule. In some embodiments, the one or more amino acid recognition molecules include at least a first amino acid binding protein comprising a first FRET label and a second amino acid binding protein comprising a second FRET label. In some embodiments, the methods further comprise detecting a series of signal pulses indicative of association of the one or more amino acid recognition molecules with the single polypeptide molecule under polypeptide degradation conditions. In some embodiments, the methods further comprise identifying a first type of amino acid in the single polypeptide molecule based on a first characteristic pattern in the series of signal pulses. In some embodiments, signal pulses of the characteristic pattern comprise a mean pulse duration of between about 1 millisecond and about 10 seconds.

In some aspects, the disclosure provides methods of identifying a terminal amino acid (e.g., the N-terminal or the C-terminal amino acid) of a polypeptide. In some embodiments, the methods comprise contacting a polypeptide with one or more labeled recognition molecules that selectively bind one or more types of terminal amino acids at a terminus of the polypeptide. In some embodiments, the methods further comprise identifying a terminal amino acid at the terminus of the polypeptide by detecting an interaction of the polypeptide with the one or more labeled recognition molecules. In some embodiments, the one or more labeled recognition molecules include at least a first amino acid binding protein comprising a first FRET label and a second amino acid binding protein comprising a second FRET label.

In yet other aspects, the disclosure provides methods of polypeptide sequencing by Edman-type degradation reactions. In some embodiments, Edman-type degradation reactions may be performed by contacting a polypeptide with different reaction mixtures for purposes of either detection or cleavage (e.g., as compared to a dynamic sequencing reaction, which can involve detection and cleavage using a single reaction mixture).

Accordingly, in some aspects, the disclosure provides methods of determining an amino acid sequence of a polypeptide comprising (i) contacting a polypeptide with one or more labeled recognition molecules that selectively bind one or more types of terminal amino acids at a terminus of the polypeptide. In some embodiments, the methods further comprise (ii) identifying a terminal amino acid (e.g., the N-terminal or the C-terminal amino acid) at the terminus of the polypeptide by detecting an interaction of the polypeptide with the one or more labeled recognition molecules. In some embodiments, the methods further comprise (iii) removing the terminal amino acid. In some embodiments, the methods further comprise (iv) repeating (i)-(iii) one or more times at the terminus of the polypeptide to determine an amino acid sequence of the polypeptide. In some embodiments, the one or more labeled recognition molecules include at least a first amino acid binding protein comprising a first FRET label and a second amino acid binding protein comprising a second FRET label.

In some embodiments, the methods further comprise, after (i) and before (ii), removing any of the one or more labeled recognition molecules that do not selectively bind the terminal amino acid. In some embodiments, the methods further comprise, after (ii) and before (iii), removing any of the one or more labeled recognition molecules that selectively bind the terminal amino acid.

In some embodiments, removing a terminal amino acid (e.g., (iii)) comprises modifying the terminal amino acid by contacting the terminal amino acid with an isothiocyanate (e.g., phenyl isothiocyanate), and contacting the modified terminal amino acid with a protease that specifically binds and removes the modified terminal amino acid. In some embodiments cleaving a terminal amino acid (e.g., (iii)) comprises modifying the terminal amino acid by contacting the terminal amino acid with an isothiocyanate, and subjecting the modified terminal amino acid to acidic or basic conditions sufficient to remove the modified terminal amino acid.

In some embodiments, identifying a terminal amino acid comprises identifying the terminal amino acid as being one type of the one or more types of terminal amino acids to which the one or more labeled recognition molecules bind. In some embodiments, identifying a terminal amino acid comprises identifying the terminal amino acid as being a type other than the one or more types of terminal amino acids to which the one or more labeled recognition molecules bind.

In some aspects, the disclosure provides methods of identifying a protein of interest in a mixed sample. In some embodiments, the methods comprise cleaving a mixed protein sample to produce a plurality of polypeptide fragments. In some embodiments, the methods further comprise determining an amino acid sequence of at least one polypeptide fragment of the plurality in a method in accordance with the methods of the disclosure. In some embodiments, the methods further comprise identifying a protein of interest in the mixed sample if the amino acid sequence is uniquely identifiable to the protein of interest.

In some embodiments, methods of identifying a protein of interest in a mixed sample comprise cleaving a mixed protein sample to produce a plurality of polypeptide fragments. In some embodiments, the methods further comprise determining amino acid sequence information from single polypeptide molecules in the plurality of polypeptide fragments in accordance with a method of polypeptide sequencing described herein. In some embodiments, the methods further comprise identifying a protein of interest in the mixed sample if the amino acid sequence is uniquely identifiable to the protein of interest.

Accordingly, in some embodiments, a polypeptide molecule or protein of interest to be analyzed in accordance with the disclosure can be of a mixed or purified sample. In some embodiments, the polypeptide molecule or protein of interest is obtained from a biological sample (e.g., blood, tissue, saliva, urine, or other biological source). In some embodiments, the polypeptide molecule or protein of interest is obtained from a patient sample (e.g., a human sample).

In some aspects, the disclosure provides systems comprising at least one hardware processor, and at least one non-transitory computer-readable storage medium storing processor-executable instructions that, when executed by the at least one hardware processor, cause the at least one hardware processor to perform a method in accordance with the disclosure. In some aspects, the disclosure provides at least one non-transitory computer-readable storage medium storing processor-executable instructions that, when executed by at least one hardware processor, cause the at least one hardware processor to perform a method in accordance with the disclosure.

The details of certain embodiments of the invention are set forth in the Detailed Description of Certain Embodiments, as described below. Other features, objects, and advantages of the invention will be apparent from the Examples, Figures, and Claims.

### BRIEF DESCRIPTION OF THE DRAWINGS

The accompanying drawings, which constitute a part of this specification, illustrate several embodiments of the invention and together with the description, serve to explain the principles of the invention.

FIG. 1 shows an example workflow for a method of polypeptide sequencing.

FIG. 2 shows an example of a dynamic peptide sequencing reaction by detection of single-molecule binding interactions.

FIGS. 3A-3E show non-limiting examples of amino acid recognition molecules labeled through a shielding element. FIG. 3A illustrates single-molecule peptide sequencing with a recognition molecule labeled through a conventional covalent linkage. FIG. 3B illustrates single-molecule peptide sequencing with a recognition molecule comprising a shielding element. FIGS. 3C-3E illustrate various examples of shielding elements in accordance with the disclosure.

### DETAILED DESCRIPTION

Aspects of the disclosure relate to methods of protein sequencing and identification, methods of polypeptide sequencing and identification, methods of amino acid identification, and compositions for performing such methods. In some aspects, the disclosure relates to the discovery of labeled binding reagents and the use of such reagents in polypeptide analysis.

In some aspects, the disclosure provides amino acid recognition molecules comprising detectable labels that undergo Förster resonance energy transfer (FRET), and the use of such reagents in polypeptide sequencing. Such detectable labels are referred to herein as “FRET labels.” In some embodiments, a FRET label comprises at least two chromophores that engage in FRET such that at least a portion of the energy absorbed by at least one donor chromophore is transferred to at least one acceptor chromophore, which emits at least a portion of the transferred energy as a detectable signal contributing to an emission spectrum. In some embodiments, at least two chromophores in a FRET label emit detectable signals that contribute to a resulting emission spectrum comprising at least two peaks.

The use of FRET labels allows for a high degree of flexibility in choosing the excitation and emission spectra for the labeled recognition molecules described herein, and provides particular advantages for differentially labeling various components of a polypeptide sequencing reaction. In particular, the use of fewer excitation light sources (e.g., a single excitation light source) dramatically reduces engineering constraints for excitation/detection systems, and also provides a more uniform analog structure to potentially provide more predictability and/or uniformity for any biochemistry steps involved in the processes. For example, in certain embodiments across a variety of different recognition molecules, one can utilize a single type of donor chromophore that has a single excitation wavelength, but couple it with multiple different acceptor chromophores (e.g., having an excitation wavelength that at least partially overlaps with the emission spectrum of the donor), where each different acceptor chromophore has an identifiably different emission spectrum. The donor chromophore may be on the same or a different recognition molecule as the acceptor chromophore. For example, in some embodiments, the donor chromophore is attached to a reaction component that interacts with multiple other reaction components, each of which can carry a detectably different acceptor chromophore. Alternatively, different donor chromophores whose emission spectra overlap may be coupled with different acceptor chromophores.

In some embodiments, the donor and acceptor chromophores are the same for multiple labeled recognition molecules, but the configuration of the labeled recognition molecule varies, resulting in a different FRET efficiency for each pair of chromophores in each labeled recognition molecule. The emission spectra from each FRET label can thereby be distinctive from every other, e.g., based on emission intensity at a plurality of emission wavelengths, as described herein. By way of illustration, a composition can comprise two labeled recognition molecules, both with the same FRET pair comprising a donor chromophore that emits at a first wavelength and an acceptor chromophore that emits at a second wavelength, where the configuration of the first labeled recognition molecule results in a FRET efficiency of 25% and the configuration of the second labeled recognition molecule results in a FRET efficiency of 75%. Under excitation illumination, the FRET pair in the first labeled recognition molecule would produce an emission spectrum with a large peak (high emission intensity) at the first wavelength and a small peak (low emission intensity) at the second wavelength, while the FRET pair in the second labeled recognition molecule would produce an emission spectrum with a small peak at the first wavelength and a large peak at the second wavelength. As such, even though both emission spectra comprise peaks at both the first and second wavelengths, these two emission spectra are distinguishable from one another, thereby allowing identification of the amino acid to which each labeled recognition molecule binds. Likewise, the same two chromophores can be used in additional labeled recognition molecules having different FRET efficiencies that result in spectra that are distinguishable from those of the first and second labeled recognition molecules, such as a FRET efficiency that results in comparable peaks at the two wavelengths.

In some embodiments, a donor chromophore can be present on a first amino acid recognition molecule and an acceptor chromophore can be present on a polypeptide. In this way, association of the first amino acid recognition molecule with the polypeptide brings the chromophores into such proximity as to permit FRET at a first efficiency, e.g., resulting in detectable emissions from both the donor and acceptor chromophores. Further, in some embodiments, a second amino acid recognition molecule comprising the donor chromophore and capable of binding to the polypeptide can also be present, where the configuration of the donor chromophore on the second amino acid recognition molecule is different than the configuration of the acceptor chromophore on the first amino acid recognition molecule. As such, binding of the second amino acid recognition molecule to the polypeptide permits FRET at a second efficiency that is different from the first, and the differing configuration of the first and second amino acid recognition molecules and resulting different FRET efficiencies upon binding the polypeptide allows identification of the amino acid bound based upon the resulting emission spectrum.

In some embodiments, compositions of the disclosure include a plurality of FRET-labeled recognition molecules having distinct emission spectra, even in embodiments in which they comprise the same set of chromophores. For example, although two FRET-labeled recognition molecules may contain the same two or more chromophores and emit at the same wavelengths, they are configured such that the emission intensities at those wavelengths are different and can be used to distinguish between the two FRET-labeled recognition molecules. In some embodiments, such differences in emission intensities is due at least in part to differing FRET efficiencies in the two FRET-labeled recognition molecules, which typically differ by at least about 5%, 10%, 15%, 20%, 25%, 30%, 40%, 50%, 60%, 70%, 80%, or 90%. For example, if one FRET-labeled recognition molecule has a FRET efficiency of 25% and a second FRET-labeled recognition molecule has a FRET efficiency of 75%, they differ by 50% FRET efficiency.

In some embodiments, a desired FRET efficiency of a FRET-labeled recognition molecule is generally 95% or less of a maximal FRET efficiency. In some embodiments, a desired FRET efficiency is between about 5% and about 95% (e.g., 10-95%, 15-95%, 20-95%, 25-95%, 30-95%, 40-95%, 50-95%, 60-95%, 70-95%, 80-95%, 90-95%, 20-80%, 25-75%, 25-50%, 50-75%) of a maximal FRET efficiency. In some embodiments, a desired FRET efficiency is selected from the group consisting of: 0%, 5%, 10%, 15%, 20%, 25%, 30%, 35%, 40%, 45%, 50%, 55%, 60%, 65%, 70%, 75%, 80%, 85%, 90%, or 95% of a maximal FRET efficiency.

In some embodiments, chromophores of a FRET label are attached to an amino acid recognition molecule (e.g., an amino acid binding protein) in a particular configuration to achieve a desired efficiency of the energy transfer between donor and acceptor chromophores, where the desired efficiency is chosen to ensure a desired emission intensity or range thereof at one or more emission wavelengths. In some embodiments, more than one such labeled recognition molecule is present in a single reaction mixture. In some embodiments, each labeled recognition molecule has an emission spectrum that is distinguishable from the emission spectrum of every other labeled recognition molecule in the mixture such that the identity of each recognition molecule can be determined. In some embodiments, the emission spectra of at least two types of labeled recognition molecules in a reaction mixture are distinguishable from one another due to variations in emission intensity at one or more wavelengths as a result of variations in FRET efficiency. In some embodiments, the multiple different labeled recognition molecules comprise the same set of chromophores in different configurations which produce different emission spectra based at least in part on different FRET efficiencies. In some embodiments, one or more non-FRET-labeled recognition molecules also present in the reaction mixture have emission spectra that are distinct from the emission spectra of the FRET-labeled recognition molecules.

In some aspects, the disclosure provides a composition comprising at least one FRET-labeled recognition molecule and at least one non-FRET-labeled recognition molecule, and methods of polypeptide sequencing using such compositions. In some aspects, the disclosure provides a composition comprising at least two FRET-labeled recognition molecules, and methods of polypeptide sequencing using such compositions. In some embodiments, such methods comprise contacting a polypeptide with the composition, detecting an emission signal indicative of association of the recognition molecules with the polypeptide, and determining amino acid sequence information from the polypeptide based on differences in emission intensity. In some embodiments, amino acid sequence information is determined using only emission intensity. For example, in some embodiments, an interaction between each type of amino acid recognition molecule and an amino acid produces a detectable emission having an emission intensity associated with the identity of the amino acid.

In some aspects, the disclosure provides compositions comprising at least two types of labeled amino acid recognition molecules, where each type binds the same type of amino acid and comprises a different type of label. For example, in some embodiments, the composition comprises a first amino acid binding protein comprising a first label, and a second amino acid binding protein comprising a second label, where the first label is different from the second label, and where the first and second amino acid binding proteins bind the same type of amino acid or subset of types of amino acids. Such compositions can be used in a dynamic polypeptide sequencing reaction to provide increased confidence levels in determining the identity of an amino acid of a polypeptide. For example, where a characteristic pattern in a series of signal pulses can be used to identify a type of amino acid in a polypeptide, the differing luminescence properties of the different labels can provide an additional identifying characteristic.

In some embodiments, a first amino acid recognition molecule comprising a first luminescent label interacts with an amino acid or a subset of amino acids, and a second amino acid recognition molecule comprising a second luminescent label interacts with the same amino acid or subset of amino acids as the first amino acid recognition molecule. In some embodiments, the first luminescent label and the second luminescent label are different and emit energy at different emission intensities and/or wavelengths. In some embodiments, detection of the first emission intensity and the second emission intensity indicate the presence of the same amino acid or subset of amino acids.

In some embodiments, a first amino acid recognition molecule comprising a first luminescent label interacts with a first amino acid or a first subset of amino acids, and a second amino acid recognition molecule comprising a second luminescent label interacts with a second amino acid or a second subset of amino acids. In some embodiments, the first amino acid or first subset of amino acids and the second amino acid or second subset of amino acids are different. In some embodiments, the first luminescent label and the second luminescent label are different and emit energy at different emission intensities and/or wavelengths. In some embodiments, detection of the first emission intensity and detection of the second emission intensity indicate the presence of the two different amino acids or subsets of amino acids.

As described herein, in some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells.

As described herein, in some embodiments, single-molecule sequencing reactions are performed in a reaction mixture comprising a binding means (e.g., one or more amino acid recognition molecules) and a cleaving means (e.g., one or more cleaving reagents). In some embodiments, reaction mixtures are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

### Dynamic Polypeptide Sequencing

In addition to methods of identifying a terminal amino acid of a polypeptide, the disclosure provides methods of sequencing polypeptides using labeled recognition molecules. In some embodiments, methods of sequencing may involve subjecting a polypeptide terminus to repeated cycles of terminal amino acid detection and terminal amino acid cleavage. For example, in some embodiments, the disclosure provides a method of determining an amino acid sequence of a polypeptide comprising contacting a polypeptide with one or more labeled recognition molecules described herein and subjecting the polypeptide to Edman degradation.

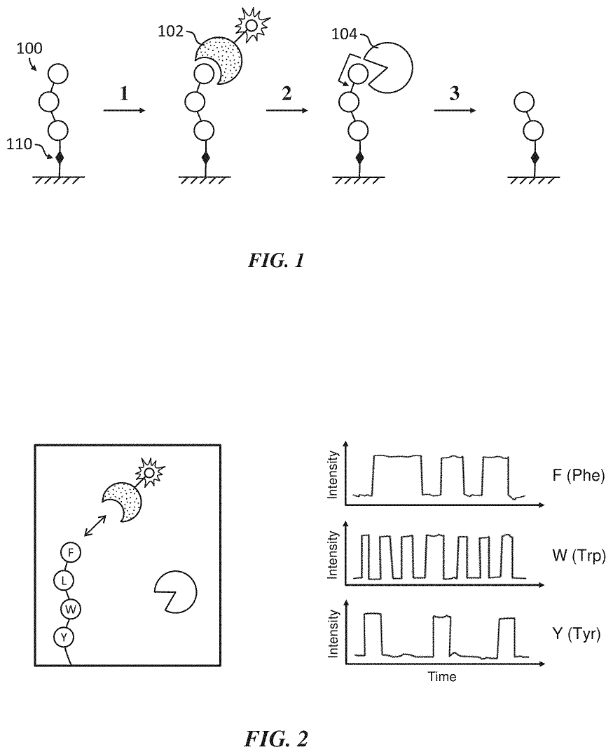

As described herein, in some aspects, the disclosure provides compositions and methods for polypeptide sequencing. FIG. 1 shows an example of a general workflow for a polypeptide sequencing reaction. As shown, in some embodiments, a polypeptide 100 is immobilized to a surface of a solid support (e.g., attached to a bottom or sidewall surface of a sample well) through a linkage group 110. In some embodiments, linkage group 110 is formed by a covalent or non-covalent linkage between a functionalized terminal end of polypeptide 100 and a complementary functional moiety attached to the surface. For example, in some embodiments, linkage group 110 is formed by a non-covalent linkage between a biotin moiety of polypeptide 100 and an avidin protein that is covalently or non-covalently attached to the surface. In some embodiments, linkage group 110 comprises a nucleic acid. Examples of linkage groups are described in detail herein.

As shown in FIG. 1, polypeptide 100 is immobilized to the surface through one terminal end such that the other terminal end is free for detecting and cleaving of a terminal amino acid in a sequencing reaction. Accordingly, in some embodiments, the reagents used in certain polypeptide sequencing reactions preferentially interact with terminal amino acids at the non-immobilized (e.g., free) terminus of polypeptide 100. In this way, polypeptide 100 remains immobilized over repeated cycles of detecting and cleaving, e.g., as in a dynamic polypeptide sequencing reaction.

In some embodiments, as shown in FIG. 1, polypeptide sequencing can proceed by (1) contacting polypeptide 100 with one or more amino acid recognition molecules that associate with one or more types of terminal amino acids. As shown, in some embodiments, a labeled amino acid recognition molecule 102 interacts with polypeptide 100 by associating with (e.g., binding to) the terminal amino acid.

In some embodiments, the method further comprises identifying the terminal amino acid of polypeptide 100 by detecting labeled amino acid recognition molecule 102 during an association event between labeled amino acid recognition molecule 102 and the terminal amino acid of polypeptide 100. In some embodiments, detecting comprises detecting a luminescence from labeled amino acid recognition molecule 102. In some embodiments, the luminescence is uniquely associated with labeled amino acid recognition molecule 102, and the luminescence is thereby associated with the type of amino acid to which labeled amino acid recognition molecule 102 binds. As such, in some embodiments, the type of amino acid is identified by determining one or more luminescence properties of labeled amino acid recognition molecule 102.

In some embodiments, polypeptide sequencing proceeds by (2) removing the terminal amino acid by contacting polypeptide 100 with a cleaving reagent 104 that binds and cleaves the terminal amino acid of polypeptide 100. In some embodiments, cleaving reagent 104 is a peptidase (e.g., an exopeptidase). Upon removal of the terminal amino acid by cleaving reagent 104, polypeptide sequencing proceeds by (3) subjecting polypeptide 100 (having n-1 amino acids) to additional cycles of terminal amino acid recognition and cleavage. In some embodiments, steps (1) through (3) occur in the same reaction mixture, e.g., as in a dynamic peptide sequencing reaction. In some embodiments, steps (1) through (3) may be carried out using other methods known in the art, such as peptide sequencing by Edman degradation.

In some embodiments, peptide sequencing can be carried out in a dynamic peptide sequencing reaction. In some embodiments, referring again to FIG. 1, the reagents required to perform steps (1) and (2) are combined within a single reaction mixture. For example, in some embodiments, steps (1) and (2) can occur without exchanging one reaction mixture for another and without a washing step as in conventional Edman degradation. Thus, in this embodiments, a single reaction mixture comprises labeled amino acid recognition molecule 102 and cleaving reagent 104. In some embodiments, cleaving reagent 104 is present in the mixture at a concentration that is less than that of labeled amino acid recognition molecule 102. In some embodiments, cleaving reagent 104 binds polypeptide 100 with a binding affinity that is less than that of labeled amino acid recognition molecule 102.

In some embodiments, dynamic polypeptide sequencing is carried out in real-time by evaluating binding interactions of labeled amino acid recognition molecules with a terminus of a polypeptide while the polypeptide is being degraded by a cleaving reagent. FIG. 2 shows an example of a method of dynamic polypeptide sequencing in which discrete binding events give rise to signal pulses of a signal output. The inset panel (left) of FIG. 2 illustrates a general scheme of real-time sequencing by this approach. As shown, a labeled amino acid recognition molecule associates with (e.g., binds to) and dissociates from a terminal amino acid (shown here as phenylalanine), which gives rise to a series of pulses in signal output which may be used to identify the terminal amino acid. In some embodiments, the series of pulses provide a pulsing pattern (e.g., a characteristic pattern) which may be diagnostic of the identity of the corresponding terminal amino acid.

As further shown in the inset panel (left) of FIG. 2, in some embodiments, a sequencing reaction mixture further comprises a cleaving reagent (e.g., an exopeptidase). In some embodiments, the exopeptidase is present in the mixture at a concentration that is less than that of the labeled amino acid recognition molecule. In some embodiments, the exopeptidase displays broad specificity such that it cleaves most or all types of terminal amino acids. Accordingly, a dynamic sequencing approach can involve monitoring recognition molecule binding at a terminus of a polypeptide over the course of a degradation reaction catalyzed by exopeptidase cleavage activity.

FIG. 2 further shows the progress of signal output intensity over time (right panels). In some embodiments, terminal amino acid cleavage by exopeptidase(s) occurs with lower frequency than the binding pulses of a labeled amino acid recognition molecule. In this way, amino acids of a polypeptide may be sequentially identified in a real-time sequencing process. In some embodiments, one type of amino acid recognition molecule can associate with more than one type of amino acid, where different characteristic patterns correspond to the association of one type of labeled amino acid recognition molecule with different types of terminal amino acids. For example, in some embodiments, different characteristic patterns (as illustrated by each of phenylalanine (F, Phe), tryptophan (W, Trp), and tyrosine (Y, Tyr)) correspond to the association of one type of labeled amino acid recognition molecule (e.g., ClpS protein) with different types of terminal amino acids over the course of degradation. In some embodiments, a plurality of labeled amino acid recognition molecules may be used, each capable of associating with different subsets of amino acids.

In some embodiments, dynamic peptide sequencing is performed by observing different association events, e.g., association events between an amino acid recognition molecule and an amino acid at a terminal end of a peptide, where each association event produces a change in magnitude of a signal, e.g., a luminescence signal, that persists for a duration of time. In some embodiments, observing different association events, e.g., association events between an amino acid recognition molecule and an amino acid at a terminal end of a peptide, can be performed during a peptide degradation process. In some embodiments, a transition from one characteristic signal pattern to another is indicative of amino acid cleavage (e.g., amino acid cleavage resulting from peptide degradation). In some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic signal patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

Methods and compositions for performing dynamic sequencing are described more fully in PCT International Application No. PCT/US2019/061831, filed Nov. 15, 2019, and PCT International Application No. PCT/US2021/033493, filed May 20, 2021, each of which is incorporated herein by reference in its entirety.

Accordingly, in some embodiments, polypeptide sequencing is performed by detecting a series of signal pulses indicative of association of one or more amino acid recognition molecules with successive amino acids exposed at the terminus of a polypeptide in an ongoing degradation reaction. The series of signal pulses can be analyzed to determine characteristic patterns in the series of signal pulses, and the time course of characteristic patterns can be used to determine an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognition molecule to cleaving reagent, ratio of one recognition molecule to another, ratio of one cleaving reagent to another), number of different reagent types (e.g., the number of different types of recognition molecules and/or cleaving reagents, the number of recognition molecule types relative to the number of cleaving reagent types), cleavage activity (e.g., peptidase activity), binding properties (e.g., kinetic and/or thermodynamic binding parameters for recognition molecule binding), reagent modification (e.g., polyol and other protein modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, a polypeptide sequencing reaction in accordance with the disclosure is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. In some embodiments, a polypeptide sequencing reaction is performed in a reaction mixture at a pH of between about 6.5 and about 9.0. In some embodiments, a polypeptide sequencing reaction is performed in a reaction mixture at a pH of between about 7.0 and about 8.5 (e.g., between about 7.0 and about 8.0, between about 7.5 and about 8.5, between about 7.5 and about 8.0, or between about 8.0 and about 8.5).

In some embodiments, a polypeptide sequencing reaction is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino)propanesulfonic acid).

In some embodiments, a polypeptide sequencing reaction is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in a reaction mixture include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, a polypeptide sequencing reaction is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

In some embodiments, polypeptide sequencing in accordance with the disclosure may be carried out by contacting a polypeptide with a sequencing reaction mixture comprising one or more amino acid recognition molecules and/or one or more cleaving reagents (e.g., peptidases). In some embodiments, a sequencing reaction mixture comprises an amino acid recognition molecule at a concentration of between about 10 nM and about 10 μM. In some embodiments, a sequencing reaction mixture comprises a cleaving reagent at a concentration of between about 500 nM and about 500 μM.

In some embodiments, a sequencing reaction mixture comprises an amino acid recognition molecule at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a sequencing reaction mixture comprises an amino acid recognition molecule at a concentration of about 100 nM, about 250 nM, about 500 nM, about 750 nM, or about 1 μM.

In some embodiments, a sequencing reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a sequencing reaction mixture comprises a cleaving reagent at a concentration of about 1 μM, about 5 μM, about 10 μM, about 30 μM, about 50 μM, about 70 μM, or about 100 μM.

In some embodiments, a sequencing reaction mixture comprises an amino acid recognition molecule at a concentration of between about 10 nM and about 10 μM, and a cleaving reagent at a concentration of between about 500 nM and about 500 μM. In some embodiments, a sequencing reaction mixture comprises an amino acid recognition molecule at a concentration of between about 100 nM and about 1 μM, and a cleaving reagent at a concentration of between about 1 μM and about 100 μM. In some embodiments, a sequencing reaction mixture comprises an amino acid recognition molecule at a concentration of between about 250 nM and about 1 μM, and a cleaving reagent at a concentration of between about 10 μM and about 100 μM. In some embodiments, a sequencing reaction mixture comprises an amino acid recognition molecule at a concentration of about 500 nM, and a cleaving reagent at a concentration of between about 25 μM and about 75 μM. In some embodiments, the concentration of an amino acid recognition molecule and/or the concentration of a cleaving reagent in a reaction mixture is as described elsewhere herein.

In some embodiments, a sequencing reaction mixture comprises an amino acid recognition molecule and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a sequencing reaction mixture comprises an amino acid recognition molecule and a cleaving reagent in a molar ratio of between about 10:1 and about 200:1. In some embodiments, a sequencing reaction mixture comprises an amino acid recognition molecule and a cleaving reagent in a molar ratio of between about 50:1 and about 150:1. In some embodiments, the molar ratio of an amino acid recognition molecule to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognition molecule to a cleaving reagent in a reaction mixture is between about 1:100 and about 1:1 or between about 1:1 and about 10:1. In some embodiments, the molar ratio of an amino acid recognition molecule to a cleaving reagent in a reaction mixture is as described elsewhere herein.

In some embodiments, a sequencing reaction mixture comprises one or more amino acid recognition molecules and one or more cleaving reagents. In some embodiments, a sequencing reaction mixture comprises at least three amino acid recognition molecules and at least one cleaving reagent. In some embodiments, the sequencing reaction mixture comprises two or more cleaving reagents. In some embodiments, the sequencing reaction mixture comprises at least one and up to ten cleaving reagents (e.g., 1-3 cleaving reagents, 2-10 cleaving reagents, 1-5 cleaving reagents, 3-10 cleaving reagents). In some embodiments, the sequencing reaction mixture comprises at least three and up to thirty amino acid recognition molecules (e.g., between 3 and 25, between 3 and 20, between 3 and 10, between 3 and 5, between 5 and 30, between 5 and 20, between 5 and 10, or between 10 and 20, amino acid recognition molecules).

In some embodiments, a sequencing reaction mixture comprises more than one amino acid recognition molecule and/or more than one cleaving reagent. In some embodiments, a sequencing reaction mixture described as comprising more than one amino acid recognition molecule (or cleaving reagent) refers to the mixture as having more than one type of amino acid recognition molecule (or cleaving reagent). For example, in some embodiments, a sequencing reaction mixture comprises two or more amino acid binding proteins. In some embodiments, the two or more amino acid binding proteins refer to two or more types of amino acid binding proteins. In some embodiments, one type of amino acid binding protein has an amino acid sequence that is different from another type of amino acid binding protein in the reaction mixture. In some embodiments, one type of amino acid binding protein has a label that is different from a label of another type of amino acid binding protein in the reaction mixture. In some embodiments, one type of amino acid binding protein associates with (e.g., binds to) a type of amino acid that is different from a type of amino acid with which another type of amino acid binding protein in the reaction mixture associates. In some embodiments, one type of amino acid binding protein associates with (e.g., binds to) a type of amino acid that is the same as a type of amino acid with which another type of amino acid binding protein in the reaction mixture associates. In some embodiments, one type of amino acid binding protein associates with (e.g., binds to) a subset of amino acids that is different from a subset of amino acids with which another type of amino acid binding protein in the reaction mixture associates. In some embodiments, one type of amino acid binding protein associates with (e.g., binds to) a subset of amino acids that at least partially (and, in some cases, entirely) overlaps with a subset of amino acids with which another type of amino acid binding protein in the reaction mixture associates.

### Amino Acid Recognition Molecules

In some embodiments, methods provided herein comprise contacting a polypeptide with an amino acid recognition molecule (also referred to herein as an amino acid binding protein), which may or may not comprise a label, that selectively binds at least one type of terminal amino acid. As used herein, in some embodiments, a terminal amino acid may refer to an amino-terminal amino acid of a polypeptide or a carboxy-terminal amino acid of a polypeptide. In some embodiments, a labeled recognition molecule selectively binds one type of terminal amino acid over other types of terminal amino acids. In some embodiments, a labeled recognition molecule selectively binds one type of terminal amino acid over an internal amino acid of the same type. In yet other embodiments, a labeled recognition molecule selectively binds one type of amino acid at any position of a polypeptide, e.g., the same type of amino acid as a terminal amino acid and an internal amino acid. In some embodiments, a labeled recognition molecule selectively binds two or more (e.g., three or more, four or more, five or more, etc.) types of amino acids over other types of amino acids.

As used herein, in some embodiments, a type of amino acid refers to one of the twenty naturally occurring amino acids or a subset of types thereof. In some embodiments, a type of amino acid refers to a modified variant of one of the twenty naturally occurring amino acids or a subset of unmodified and/or modified variants thereof. Examples of modified amino acid variants include, without limitation, post-translationally-modified variants (e.g., acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination), chemically modified variants, unnatural amino acids, and proteinogenic amino acids such as selenocysteine and pyrrolysine. In some embodiments, a subset of types of amino acids includes more than one and fewer than twenty amino acids having one or more similar biochemical properties. For example, in some embodiments, a type of amino acid refers to one type selected from amino acids with charged side chains (e.g., positively and/or negatively charged side chains), amino acids with polar side chains (e.g., polar uncharged side chains), amino acids with nonpolar side chains (e.g., nonpolar aliphatic and/or aromatic side chains), and amino acids with hydrophobic side chains.

In some embodiments, methods provided herein comprise contacting a polypeptide with one or more labeled recognition molecules that selectively bind one or more types of terminal amino acids. As an illustrative and non-limiting example, where four labeled recognition molecules are used in a method of the disclosure, any one recognition molecule selectively binds one type of terminal amino acid that is different from another type of amino acid to which any of the other three selectively binds (e.g., a first recognition molecule binds a first type, a second recognition molecule binds a second type, a third recognition molecule binds a third type, and a fourth recognition molecule binds a fourth type of terminal amino acid). For the purposes of this discussion, one or more labeled recognition molecules in the context of a method described herein may be alternatively referred to as a set of labeled recognition molecules.

In some embodiments, a set of labeled recognition molecules comprises at least one and up to six labeled recognition molecules. For example, in some embodiments, a set of labeled recognition molecules comprises one, two, three, four, five, or six labeled recognition molecules. In some embodiments, a set of labeled recognition molecules comprises ten or fewer labeled recognition molecules. In some embodiments, a set of labeled recognition molecules comprises eight or fewer labeled recognition molecules. In some embodiments, a set of labeled recognition molecules comprises six or fewer labeled recognition molecules. In some embodiments, a set of labeled recognition molecules comprises four or fewer labeled recognition molecules. In some embodiments, a set of labeled recognition molecules comprises three or fewer labeled recognition molecules. In some embodiments, a set of labeled recognition molecules comprises two or fewer labeled recognition molecules. In some embodiments, a set of labeled recognition molecules comprises four labeled recognition molecules. In some embodiments, a set of labeled recognition molecules comprises at least two and up to twenty (e.g., at least two and up to ten, at least two and up to eight, at least four and up to twenty, at least four and up to ten) labeled recognition molecules. In some embodiments, a set of labeled recognition molecules comprises more than twenty (e.g., 20 to 25, 20 to 30) recognition molecules. It should be appreciated, however, that any number of recognition molecules may be used in accordance with a method of the disclosure to accommodate a desired use.

In accordance with the disclosure, in some embodiments, one or more types of amino acids are identified by detecting luminescence of a labeled recognition molecule. In some embodiments, a labeled recognition molecule comprises a recognition molecule that selectively binds one type of amino acid and a luminescent label having a luminescence that is associated with the recognition molecule. In this way, the luminescence (e.g., luminescence lifetime, luminescence intensity, and other luminescence properties described elsewhere herein) may be associated with the selective binding of the recognition molecule to identify an amino acid of a polypeptide. In some embodiments, a plurality of types of labeled recognition molecules may be used in a method according to the disclosure, where each type comprises a luminescent label having a luminescence that is uniquely identifiable from among the plurality. In some embodiments, the luminescent label of each type of labeled recognition molecule is uniquely identifiable from among the plurality by luminescence intensity alone. Suitable luminescent labels may include luminescent molecules, such as fluorophore dyes, and are described elsewhere herein.

In some embodiments, an amino acid recognition molecule may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide. In some embodiments, desirable properties include an ability to bind selectively and with low affinity (e.g., with a K_{D} of about 50 nM or higher, for example, between about 50 nM and about 50 μM, between about 100 nM and about 10 μM, between about 500 nM and about 50 μM) to more than one type of amino acid. For example, in some aspects, the disclosure provides methods of sequencing by detecting reversible binding interactions during a polypeptide degradation process. Advantageously, such methods may be performed using a recognition molecule that reversibly binds with low affinity to more than one type of amino acid (e.g., a subset of amino acid types).

As used herein, in some embodiments, the terms “selective” and “specific” (and variations thereof, e.g., selectively, specifically, selectivity, specificity) refer to a preferential binding interaction. For example, in some embodiments, an amino acid recognition molecule that selectively binds one type of amino acid preferentially binds the one type over another type of amino acid. A selective binding interaction will discriminate between one type of amino acid (e.g., one type of terminal amino acid) and other types of amino acids (e.g., other types of terminal amino acids), typically more than about 10- to 100-fold or more (e.g., more than about 1,000- or 10,000-fold). Accordingly, it should be appreciated that a selective binding interaction can refer to any binding interaction that is uniquely identifiable to one type of amino acid over other types of amino acids. For example, in some aspects, the disclosure provides methods of polypeptide sequencing by obtaining data indicative of association of one or more amino acid recognition molecules with a polypeptide molecule. In some embodiments, the data comprises a series of signal pulses corresponding to a series of reversible amino acid recognition molecule binding interactions with an amino acid of the polypeptide molecule, and the data may be used to determine the identity of the amino acid. As such, in some embodiments, a “selective” or “specific” binding interaction refers to a detected binding interaction that discriminates between one type of amino acid and other types of amino acids.

In some embodiments, an amino acid recognition molecule binds one type of amino acid with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other types of amino acids. In some embodiments, an amino acid recognition molecule binds one type of amino acid (e.g., one type of terminal amino acid) with a K_{D} of less than about 100 nM, less than about 50 nM, less than about 25 nM, less than about 10 nM, or less than about 1 nM. In some embodiments, an amino acid recognition molecule binds one type of amino acid with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds one type of amino acid with a K_{D} of about 50 nM.

In some embodiments, an amino acid recognition molecule binds two or more types of amino acids with a K_{D} of less than about 10⁻⁶ M (e.g., less than about 10⁻⁷ M, less than about 10⁻⁸ M, less than about 10⁻⁹ M, less than about 10⁻¹⁰ less than about 10⁻¹¹ M, less than about 10⁻¹² M, M, to as low as 10⁻¹⁶ M). In some embodiments, an amino acid recognition molecule binds two or more types of amino acids with a K_{D} of less than about 100 nM, less than about 50 nM, less than about 25 nM, less than about 10 nM, or less than about 1 nM. In some embodiments, an amino acid recognition molecule binds two or more types of amino acids with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds two or more types of amino acids with a K_{D} of about 50 nM.

In some embodiments, an amino acid recognition molecule binds at least one type of amino acid with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some embodiments, the value for K_{D} or koff can be a known literature value, or the value can be determined empirically. In some embodiments, the value for koff can be determined empirically based on signal pulse information obtained in a single-molecule assay as described elsewhere herein. For example, the value for koff can be approximated by the reciprocal of the mean pulse duration. In some embodiments, an amino acid recognition molecule binds two or more types of amino acids with a different K_{D} or koff for each of the two or more types. In some embodiments, a first K_{D} or koff for a first type of amino acid differs from a second K_{D} or koff for a second type of amino acid by at least 10% (e.g., at least 25%, at least 50%, at least 100%, or more). In some embodiments, the first and second values for K_{D} or koff differ by about 10-25%, 25-50%, 50-75%, 75-100%, or more than 100%, for example by about 2-fold, 3-fold, 4-fold, 5-fold, or more.

As described herein, an amino acid recognition molecule may be any biomolecule capable of selectively or specifically binding one molecule over another molecule (e.g., one type of amino acid over another type of amino acid). In some embodiments, a recognition molecule is not a peptidase or does not have peptidase activity. For example, in some embodiments, methods of polypeptide sequencing of the disclosure involve contacting a polypeptide molecule with one or more recognition molecules and a cleaving reagent. In such embodiments, the one or more recognition molecules do not have peptidase activity, and removal of one or more amino acids from the polypeptide molecule (e.g., amino acid removal from a terminus of the polypeptide molecule) is performed by the cleaving reagent.

Recognition molecules include, for example, proteins and nucleic acids, which may be synthetic or recombinant. In some embodiments, a recognition molecule may be an antibody or an antigen-binding portion of an antibody, an SH2 domain-containing protein or fragment thereof, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some aspects, the disclosure relates to the discovery and development of amino acid recognition molecules for use in accordance with methods described herein or known in the art. In some embodiments, the disclosure provides amino acid binding proteins (e.g., ClpS proteins) having binding properties that were previously not known to exist among other homologous members of a protein family. In some embodiments, the disclosure provides engineered amino acid binding proteins. For example, in some embodiments, the disclosure provides fusion constructs comprising a single polypeptide having tandem copies of two or more amino acid binding proteins.

The inventors have recognized and appreciated that fusion constructs of the disclosure allow for an effective increase in recognition molecule concentration without increasing label background noise (e.g., background fluorescence). The inventors have further recognized and appreciated that fusion constructs of the disclosure provide increased accuracy in sequencing reactions and/or decrease the amount of time required to perform a sequencing reaction. Additionally, by providing fusion constructs having tandem copies of two or more different types of amino acid binding proteins, fewer reagents are required in reactions, which provides a more efficient and inexpensive approach for sequencing.

In some embodiments, a recognition molecule of the disclosure is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, a recognition molecule is an N-end rule pathway protein selected from a Gid protein (e.g., Gid4 or Gid10 protein), a UBR-box protein (e.g., UBR1, UBR2) or UBR-box domain-containing protein fragment thereof, a p62 protein or ZZ domain-containing fragment thereof, and a ClpS protein (e.g., ClpS1, ClpS2). Accordingly, in some embodiments, a labeled recognition molecule comprises a degradation pathway protein. In some embodiments, a labeled recognition molecule comprises a ClpS protein.

In some embodiments, a recognition molecule of the disclosure is a ClpS protein, such as Agrobacterium tumifaciens ClpS1, Agrobacterium tumifaciens ClpS2, Synechococcus elongatus ClpS1, Synechococcus elongatus ClpS2, Thermosynechococcus elongatus ClpS, Escherichia coli ClpS, or Plasmodium falciparum ClpS. In some embodiments, the recognition molecule is an L/F transferase, such as Escherichia coli leucyl/phenylalanyl-tRNA-protein transferase. In some embodiments, the recognition molecule is a D/E leucyltransferase, such as Vibrio vulnificus Aspartate/glutamate leucyltransferase Bpt. In some embodiments, the recognition molecule is a UBR protein or UBR-box domain, such as the UBR protein or UBR-box domain of human UBR1 and UBR2 or Saccharomyces cerevisiae UBR1. In some embodiments, the recognition molecule is a p62 protein, such as H. sapiens p62 protein or Rattus norvegicus p62 protein, or truncation variants thereof that minimally include a ZZ domain. In some embodiments, the recognition molecule is a Gid4 protein, such as H. sapiens GID4 or Saccharomyces cerevisiae GID4. In some embodiments, the recognition molecule is a Gid10 protein, such as Saccharomyces cerevisiae GID10. In some embodiments, the recognition molecule is an N-meristoyltransferase, such as Leishmania major N-meristoyltransferase or H. sapiens N-meristoyltransferase NMT1. In some embodiments, the recognition molecule is a BIR2 protein, such as Drosophila melanogaster BIR2. In some embodiments, the recognition molecule is a tyrosine kinase or SH2 domain of a tyrosine kinase, such as H. sapiens Fyn SH2 domain, H. sapiens Src tyrosine kinase SH2 domain, or variants thereof, such as H. sapiens Fyn SH2 domain triple mutant superbinder. In some embodiments, the recognition molecule is an antibody or antibody fragment, such as a single-chain antibody variable fragment (scFv) against phosphotyrosine or another post-translationally modified amino acid variant described herein.

In some embodiments, an amino acid recognition molecule comprises a single polypeptide having tandem copies of two or more amino acid binding proteins (e.g., two or more binders). As used herein, in some embodiments, a tandem arrangement or orientation of elements in a molecule refers to an end-to-end joining of each element to the next element in a linear fashion such that the elements are fused in series. For example, in some embodiments, a polypeptide having tandem copies of two binders refers to a fusion polypeptide in which the C-terminus of one binder is fused to the N-terminus of the other binder. Similarly, a polypeptide having tandem copies of two or more binders refers to a fusion polypeptide in which the C-terminus of a first binder is fused to the N-terminus of a second binder, the C-terminus of the second binder is fused to the N-terminus of a third binder, and so forth. Such fusion polypeptides can comprise multiple copies of the same binder or multiple copies of different binders. In some embodiments, a fusion polypeptide of the disclosure has at least two and up to ten binders (e.g., at least 2 binders and up to eight, six, five, four, or three binders). In some embodiments, a fusion polypeptide of the disclosure has five or fewer binders (e.g., two, three, four, or five binders). Accordingly, in some embodiments, a labeled recognition molecule comprises a fusion polypeptide of the disclosure.

In some embodiments, a fusion polypeptide is provided by expression of a single coding sequence containing segments encoding monomeric binder subunits separated by segments encoding flexible linkers, where expression of the single coding sequence produces a single full-length polypeptide having two or more independent binding sites. In some embodiments, one or more of the monomeric subunits (e.g., binders) are ClpS proteins. In some embodiments, ClpS subunits may be identical or non-identical. Where non-identical, ClpS subunits may be distinct variants of the same parent ClpS protein, or they may be derived from different parent ClpS proteins. In some embodiments, a fusion polypeptide comprises one or more ClpS monomers and one or more non-ClpS monomers. In some embodiments, the monomeric subunits comprise non-ClpS monomers. In some embodiments, the monomeric subunits comprise one or more degradation pathway proteins. For example, in some embodiments, the monomeric subunits comprise one or more of a Gid protein, a UBR-box protein or UBR-box domain-containing protein fragment thereof, a p62 protein or ZZ domain-containing fragment thereof, and a ClpS protein (e.g., ClpS1, ClpS2).

In some embodiments, binders of a fusion polypeptide recognize the same set of one or more amino acids. In some embodiments, binders of a fusion polypeptide recognize a distinct set of one or more amino acids. In some embodiments, binders of a fusion polypeptide recognize an overlapping set of amino acids. In some embodiments, where the binders of a fusion polypeptide recognize the same amino acid, they may recognize the amino acid with the same characteristic pulsing pattern or with different characteristic pulsing patterns.

In some embodiments, binders of a fusion polypeptide are joined end-to-end, either by a covalent bond or a linker that covalently joins the C-terminus of one binder to the N-terminus of another binder. In the context of fusion polypeptides of the disclosure, a linker refers to one or more amino acids within a fusion polypeptide that joins two binders and that does not form part of the polypeptide sequence corresponding to either of the two binders. In some embodiments, a linker comprises at least two amino acids (e.g., at least 2, 3, 4, 5, 6, 8, 10, 15, 25, 50, 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 5 and about 50, between about 2 and about 20, between about 5 and about 20, or between about 2 and about 30, amino acids).

In some aspects, the disclosure provides a nucleic acid encoding a single polypeptide having tandem copies of two or more amino acid binding proteins. In some embodiments, the nucleic acid is an expression construct encoding a fusion polypeptide of the disclosure. In some embodiments, an expression construct encodes a fusion polypeptide having at least two and up to ten binders (e.g., at least 2 binders and up to eight, six, five, four, or three binders). In some embodiments, an expression construct encodes a fusion polypeptide having five or fewer binders (e.g., two, three, four, or five binders).

In some embodiments, a recognition molecule of the disclosure is an amino acid binding protein which can be used with other types of amino acid binding molecules, such as a peptidase and/or a nucleic acid aptamer, in a method of sequencing. A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, a labeled recognition molecule comprises a peptidase that has been modified to inactivate exopeptidase or endopeptidase activity. In this way, the labeled recognition molecule selectively binds without also cleaving the amino acid from a polypeptide. In yet other embodiments, a peptidase that has not been modified to inactivate exopeptidase or endopeptidase activity may be used with an amino acid binding protein of the disclosure. For example, in some embodiments, a labeled recognition molecule comprises a labeled exopeptidase.

In some embodiments, an amino acid recognition molecule comprises one or more labels. In some embodiments, the one or more labels comprise a luminescent label or a conductivity label as described elsewhere herein. In some embodiments, the one or more labels comprise one or more polyol moieties (e.g., one or more moieties selected from dextran, polyvinylpyrrolidone, polyethylene glycol, polypropylene glycol, polyoxyethylene glycol, and polyvinyl alcohol). For example, in some embodiments, an amino acid recognition molecule is PEGylated. In some embodiments, polyol modification (e.g., PEGylation) can limit the extent of non-specific sticking to a substrate (e.g., sequencing chip) surface. In some embodiments, polyol modification can limit the extent of aggregation or interaction between an amino acid recognition molecule with other recognition molecules, with a cleaving reagent, or with other species present in a sequencing reaction mixture. PEGylation can be performed by incubating a recognition molecule (e.g., an amino acid binding protein, such as a ClpS protein) with mPEG4-NHS ester, which labels primary amines such as surface-exposed lysine side chains. Other types of PEG and other methods of polyol modification are known in the art.

In some embodiments, the one or more labels comprise a tag sequence. For example, in some embodiments, an amino acid recognition molecule comprises a tag sequence that provides one or more functions other than amino acid binding. In some embodiments, a tag sequence comprises at least one biotin ligase recognition sequence that permits biotinylation of the recognition molecule (e.g., incorporation of one or more biotin molecules, including biotin and bis-biotin moieties). In some embodiments, the tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem.

Additional examples of functional sequences in a tag sequence include purification tags, cleavage sites, and other moieties useful for purification and/or modification of recognition molecules. Table 1 provides a list of non-limiting sequences of tag sequences, any one or more of which may be used in combination with any one of the amino acid recognition molecules of the disclosure (e.g., in combination with an amino acid binding protein). It should be appreciated that the tag sequences shown in Table 1 are meant to be non-limiting, and recognition molecules in accordance with the disclosure can include any one or more of the tag sequences (e.g., His-tags and/or biotinylation tags) at the N- or C-terminus of a recognition molecule polypeptide or at an internal position, split between the N- and C-terminus, or otherwise rearranged as practiced in the art.

TABLE 1
Non-limiting examples of tag sequences.

| Tag | Sequence |
|---|---|
| Biotinylation tag | GGGSGGGSGGGSGLNDFFEAQKIEWHE |
| | (SEQ ID NO: 1) |
| Bis-biotinylation | GGGSGGGSGGGSGLNDFFEAQKIEWHE |
| tag | GGGSGGGSGGGSGLNDFFEAQKIEWHE |
| | (SEQ ID NO: 2) |
| Bis-biotinylation | GSGGGSGGGSGGGSGLNDFFEAQKIEW |
| tag | HEGGGSGGGSGGGSGLNDFFEAQKIEW |
| | HE |
| | (SEQ ID NO: 3) |
| His/biotinylation | GHHHHHHHHHHGGGSGGGSGGGSGLND |
| tag | FFEAQKIEWHE |
| | (SEQ ID NO: 4) |
| His/bis- | GHHHHHHHHHHGGGSGGGSGGGSGLND |
| biotinylation tag | FFEAQKIEWHEGGGSGGGSGGGSGLND |
| | FFEAQKIEWHE |
| | (SEQ ID NO: 5) |
| His/bis- | GGSHHHHHHHHHHGGGSGGGSGGGSGL |
| biotinylation tag | NDFFEAQKIEWHEGGGSGGGSGGGSGL |
| | NDFFEAQKIEWHE |
| | (SEQ ID NO: 6) |
| His/bis- | GSHHHHHHHHHHGGGSGGGSGGGSGLN |
| biotinylation tag | DFFEAQKIEWHEGGGSGGGSGGGSGLN |
| | DFFEAQKIEWHE |
| | (SEQ ID NO: 7) |
| Bis-biotinylation/ | GGGSGGGSGGGSGLNDFFEAQKIEWHE |
| His tag | GGGSGGGSGGGSGLNDFFEAQKIEWHE |
| | GHHHHHH |
| | (SEQ ID NO: 8) |

Examples of amino acid recognition molecules (e.g., amino acid binding proteins) for use in accordance with the disclosure are described more fully in PCT International Application No. PCT/US2019/061831, filed Nov. 15, 2019, and PCT International Application No. PCT/US2021/033493, filed May 20, 2021, the relevant content of which is incorporated herein by reference in its entirety.

### Shielded Recognition Molecules

In accordance with embodiments described herein, single-molecule polypeptide sequencing methods can be carried out by illuminating a surface-immobilized polypeptide with excitation light, and detecting luminescence produced by a label attached to an amino acid recognition molecule. In some cases, radiative and/or non-radiative decay produced by the label can result in photodamage to the polypeptide.

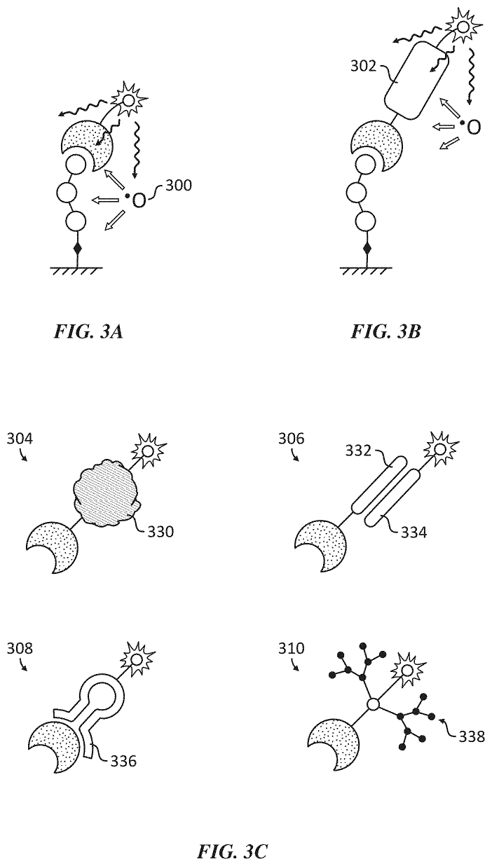

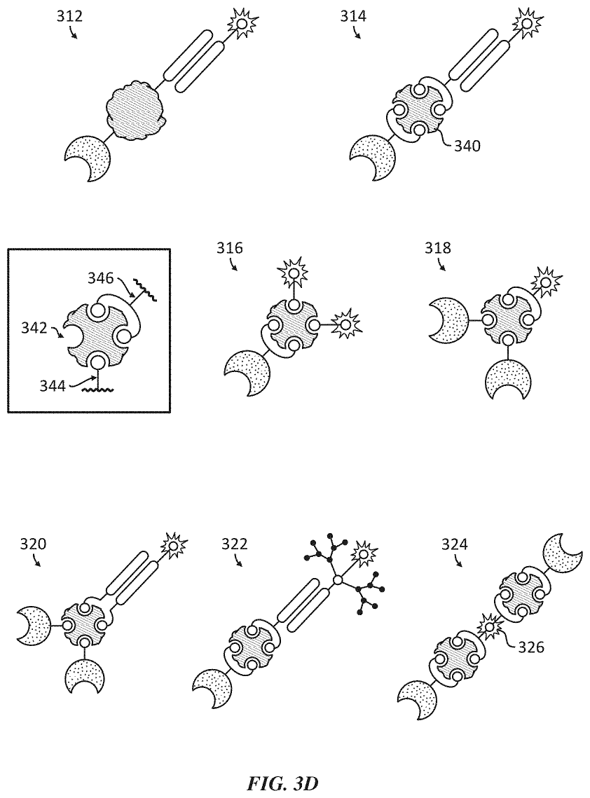

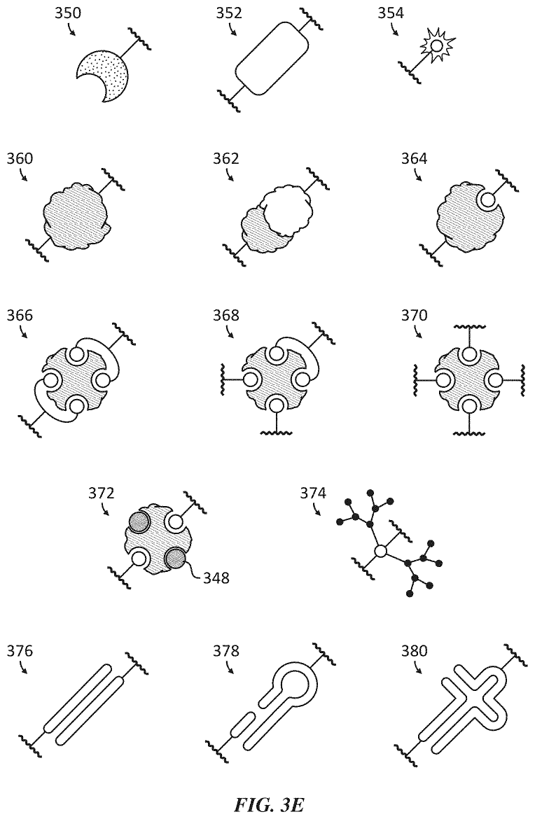

FIG. 3A illustrates an example sequencing reaction in which a recognition molecule is shown associated with a polypeptide immobilized to a surface. In the presence of excitation illumination, the label can produce fluorescence through radiative decay, which results in a detectable association event. However, in some cases, the label produces non-radiative decay, which can result in the formation of reactive oxygen species 300. The reactive oxygen species 300 can eventually damage the immobilized peptide, such that the reaction ends before obtaining complete sequence information for the polypeptide. This photodamage can occur, for example, at the exposed polypeptide terminus (top open arrow), at an internal position on the polypeptide (middle open arrow), or at the surface linkage group attaching the polypeptide to the surface (bottom open arrow). The inventors have found that photodamage can be mitigated and recognition times extended by incorporation of a shielding element into an amino acid recognition molecule.

FIG. 3B illustrates an example sequencing reaction using a shielded recognition molecule that includes a shielding element 302. Shielding element 302 forms a covalent or non-covalent linkage group that provides increased distance between the label and polypeptide, such that damaging effects from reactive oxygen species 300 can be reduced due to free radical decay over the separation distance between the label and the polypeptide. Shielding element 302 can also provide a steric barrier that shields the polypeptide from the label by absorbing damage from reactive oxygen species 300 and radiative and/or non-radiative decay.

Without wishing to be bound by theory, it is thought that a shielding element, positioned between a recognition component and a label component, can absorb, deflect, or otherwise block radiative and/or non-radiative decay emitted by the label component. In some embodiments, the shielding element prevents or limits the extent to which one or more labels (e.g., luminescent labels) interact with one or more amino acid recognition molecules. In some embodiments, the shielding element prevents or limits the extent to which one or more labels interact with one or more molecules associated with an amino acid recognition molecule (e.g., a polypeptide associated with the recognition molecule, a polypeptide surface linkage group). Accordingly, in some embodiments, the term shielding can generally refer to a protective or shielding effect that is provided by some portion of a linkage group formed between a recognition component and a label component.

In some embodiments, a shielding element, which may generally be referred to as a shield herein, is attached to one or more amino acid recognition molecules (e.g., a recognition component) and to one or more labels (e.g., a label component). In some embodiments, the recognition and label components are attached at non-adjacent sites on the shield. For example, one or more amino acid recognition molecules can be attached to a first side of the shield, and one or more labels can be attached to a second side of the shield, where the first and second sides of the shield are distant from each other. In some embodiments, the attachment sites are on approximately opposite sides of the shield.

The distance between the site at which a shield is attached to a recognition molecule and the site at which the shield is attached to a label can be a linear measurement through space or a non-linear measurement across the surface of the shield. The distance between the recognition molecule and label attachment sites on a shield can be measured by modeling the three-dimensional structure of the shield. In some embodiments, this distance can be at least 2 nm, at least 4 nm, at least 6 nm, at least 8 nm, at least 10 nm, at least 12 nm, at least 15 nm, at least 20 nm, at least 30 nm, at least 40 nm, or more. Alternatively, the relative positions of the recognition molecule and label on a shield can be described by treating the structure of the shield as a quadratic surface (e.g., ellipsoid, elliptic cylinder). In some embodiments, the recognition molecule and label attachment sites are separated by a distance that is at least one eighth of the distance around an ellipsoidal shape representing the shield. In some embodiments, the recognition molecule and label are separated by a distance that is at least one quarter of the distance around an ellipsoidal shape representing the shield. In some embodiments, the recognition molecule and label are separated by a distance that is at least one third of the distance around an ellipsoidal shape representing the shield. In some embodiments, the recognition molecule and label are separated by a distance that is one half of the distance around an ellipsoidal shape representing the shield.

The size of a shield should be such that a label is unable or unlikely to directly contact the polypeptide when the amino acid recognition molecule is associated with the polypeptide. The size of a shield should also be such that an attached label is detectable when the amino acid recognition molecule is associated with the polypeptide. For example, the size should be such that an attached luminescent label is within an illumination volume to be excited.

It should be appreciated that there are a variety of parameters by which a practitioner could evaluate shielding effects. Generally, the effects of a shielding element can be evaluated by conducting a comparative assessment between a composition having the shielding element and a composition lacking the shielding element. For example, a shielding element can increase recognition time of an amino acid recognition molecule. In some embodiments, recognition time refers to the length of time in which association events between the recognition molecule and a polypeptide are observable in a polypeptide sequencing reaction as described herein. In some embodiments, recognition time is increased by about 10-25%, 25-50%, 50-75%, 75-100%, or more than 100%, for example by about 2-fold, 3-fold, 4-fold, 5-fold, or more, relative to a polypeptide sequencing reaction performed under the same conditions, with the exception that the amino acid recognition molecule lacks the shielding element but is otherwise similar or identical. In some embodiments, a shielding element can increase sequencing accuracy and/or sequence read length (e.g., by at least 5%, at least 10%, at least 15%, at least 25% or more, relative to a sequencing reaction performed under comparative conditions as described above).

Accordingly, in some aspects, the disclosure provides shielded recognition molecules comprising at least one amino acid recognition molecule, at least one detectable label, and a shielding element that forms a covalent or non-covalent linkage group between the recognition molecule and label. In some embodiments, a shielding element is at least 2 nm, at least 5 nm, at least 10 nm, at least 12 nm, at least 15 nm, at least 20 nm, or more, in length (e.g., in an aqueous solution). In some embodiments, a shielding element is between about 2 nm and about 100 nm in length (e.g., between about 2 nm and about 50 nm, between about 10 nm and about 50 nm, between about 20 nm and about 100 nm).

In some embodiments, a shield (e.g., shielding element) forms a covalent or non-covalent linkage group between one or more amino acid recognition molecules (e.g., a recognition component) and one or more labels (e.g., a label component). As used herein, in some embodiments, covalent and non-covalent linkages or linkage groups refer to the nature of the attachments of the recognition and label components to the shield. In some embodiments, covalent and non-covalent linkages or linkage groups refer to the nature of the attachments of the chromophores within a label component (e.g., a FRET label) to the shield.

In some embodiments, a covalent linkage, or a covalent linkage group, refers to a shield that is attached to each of the recognition and label components through a covalent bond or a series of contiguous covalent bonds. Covalent attachment one or both components can be achieved by covalent conjugation methods known in the art. For example, in some embodiments, click chemistry techniques (e.g., copper-catalyzed, strain-promoted, copper-free click chemistry, etc.) can be used to attach one or both components to the shield. Such methods generally involve conjugating one reactive moiety to another reactive moiety to form one or more covalent bonds between the reactive moieties. Accordingly, in some embodiments, a first reactive moiety of a shield can be contacted with a second reactive moiety of a recognition or label component to form a covalent attachment. Examples of reactive moieties include, without limitation, reactive amines, azides, alkynes, nitrones, alkenes (e.g., cycloalkenes), tetrazines, tetrazoles, and other reactive moieties suitable for click reactions and similar coupling techniques.

In some embodiments, a non-covalent linkage, or a non-covalent linkage group, refers to a shield that is attached to one or both of the recognition and label components through one or more non-covalent coupling means, including but not limited to receptor-ligand interactions and oligonucleotide strand hybridization. Examples of receptor-ligand interactions are provided herein and include, without limitation, protein-protein complexes, protein-ligand complexes, protein-aptamer complexes, and aptamer-nucleic acid complexes. Various configurations and strategies for oligonucleotide strand hybridization are described herein and are known in the art (see, e.g., U.S. Patent Publication No. 2019/0024168).

In some aspects, the labeled amino acid recognition molecules of the disclosure are characterized by the specific distances provided between the chromophores (e.g., in a FRET pair) in the label component of a shielded recognition molecule. In some embodiments, such distances between chromophores is configured to achieve a desired luminescent property, such as a desired FRET efficiency. Accordingly, in some embodiments, a shielding element of the disclosure provides a scaffold upon which chromophores of a label component may be attached in a particular configuration.

As used herein, in some embodiments, a “configuration” in the context of a detectable label, such as chromophores of a FRET label, refers to the spatial orientation of chromophores relative to one another, relative to an amino acid recognition molecule, and/or relative to a polypeptide molecule to which the amino acid recognition molecule binds. In some embodiments, a configuration can also refer to the types of chromophores and/or the number of copies of each type of chromophore. In some embodiments, a specific configuration can be achieved by attachment of one or more chromophores to a respective one or more attachment sites on a shielding element described herein. In some embodiments, the shielding element provides a labeling scaffold that maintains a distance of about 2 nm to about 10 nm (e.g., 2-8 nm, 2-6 nm, 4-10 nm, 6-10 nm) between chromophores in a FRET pair. The specific spacing between the chromophores will vary depending on the chromophores used and the desired FRET efficiency (0-100%).

In some embodiments, the chromophores in a FRET label are configured to achieve a desired FRET efficiency, which can refer to the efficiency of the energy transfer between the donor and acceptor chromophores, where the desired FRET efficiency is chosen to ensure a desired emission intensity at one or more emission wavelengths in the emission spectrum. As used herein, emission intensity can refer to the intensity of emitted signal at a given wavelength, and can generally be related to the height of a peak in an emission spectrum graph, where a relatively higher peak is indicative of a higher emission intensity and a relatively lower peak is indicative of a lower emission intensity. FRET efficiency (E) generally refers to the loss in intensity of the donor chromophore emission in the presence of the acceptor chromophore, and can be expressed using the following equation: E=1−(F_{D}A/F_{D}), where F_{DA} is the fluorescence intensity of the donor in the presence of the acceptor and FD is the fluorescence intensity of the donor in the absence of the acceptor. The equation for FRET efficiency provides the fraction of donor fluorescence that is transferred to the acceptor fluorophore. For example, in theory, a transfer of 100% of donor fluorescence to the acceptor fluorophore would yield a value of zero for FDA, which would provide a maximal FRET efficiency of 1, or 100% (E=1−(0)=1).

In some embodiments, the configuration of the chromophores (e.g., spacing between them) in a FRET label determines the FRET efficiency, and therefore the emission spectrum. For example, in some embodiments, a FRET label comprising chromophores separated by a spacing of about 2 nm results in a relatively high FRET efficiency, while a spacing of about 9 nm results in a relatively low FRET efficiency. Other factors that influence FRET efficiency include the spectral overlap of the donor emission spectrum and the acceptor absorption spectrum, and the relative orientation of the donor emission dipole moment and the acceptor absorption dipole moment.

In some embodiments, where a FRET efficiency is less than 100%, at least two chromophores in a FRET label emit detectable signals that contribute to the resulting multi-spectral emission spectrum, e.g., represented by at least two “peaks” characterized by their wavelength and intensity. In general, as the FRET efficiency increases, the emission intensity at the emission wavelength of the donor chromophore decreases and the emission intensity at the emission wavelength of the acceptor chromophore increases. As such, two FRET labels that each comprise the same set of chromophores can have distinct emission spectra if each is configured to ensure a distinct FRET efficiency or range thereof. For example, if a first FRET label has a higher FRET efficiency than a second FRET label, the emission spectrum corresponding to the first FRET label will have a relatively lower intensity peak at the emission wavelength of the donor chromophore and a relatively higher intensity peak at the emission wavelength of the acceptor chromophore than does the second FRET label. For example, in some embodiments, the intensity of the first FRET label at the emission wavelength of the donor chromophore may be less than 90%, 80%, 70%, 60%, 50%, 40%, 30%, 20%, 10%, or 5% of that of the second FRET label; and the intensity of the second FRET label at the emission wavelength of the acceptor chromophore may be less than 90%, 80%, 70%, 60%, 50%, 40%, 30%, 20%, 10%, or 5% of that of the first FRET label. The differences between emission intensities at donor or acceptor emission wavelengths for two different FRET labels can also be expressed as ratios of the intensities for each label, e.g., 10:1, 8:1, 6:1, 4:1, 3:1, 2:1, 1:2, 1:3, 1:4, 1:6, 1:8, or 1:10 at a given wavelength. In this way, FRET labels comprising the same set of chromophores can be configured such that each has a distinctive emission spectra based at least on emission intensities, even if emission wavelengths are the same. In some embodiments, a single FRET pair may be used to provide at least about 2-10 different emission spectra based on the orientation of the chromophores relative to one another. In some embodiments, FRET labels having more than two chromophores can provide 10 or more different emission spectra based on the orientation of the chromophores with respect to one another, and therefore the relative FRET efficiencies of each transfer event within the label.

A number of shields may be employed as labeling scaffolds that will provide the desired configuration of FRET label chromophores within a FRET-labeled recognition molecule or a complex of multiple labeled molecules, e.g., including the separation between chromophores in a FRET pair, the distance between a chromophore of a FRET pair and an amino acid recognition molecule, or the distance between a chromophore in a FRET-labeled recognition molecule and a chromophore in a labeled polypeptide when the FRET-labeled recognition molecule and the labeled polypeptide are bound to or otherwise associated with one another. In general, a shielding element comprising one or more chromophores can be linear or branched, and multiple shields may be utilized in a single labeled amino acid recognition molecule. For example, a single shield may be bound to an amino acid recognition molecule, and this shield may comprise multiple attachment sites, where each attachment site comprises a single chromophore of a FRET pair, and where the orientation of the multiple attachment sites ensures a given distance between the two chromophores, thereby ensuring a desired FRET efficiency upon excitation illumination. In some embodiments, a single attachment site may contain a further shield or linkage group comprising more than one chromophore, with the further shield or linkage group designed to ensure a given orientation between the more than one chromophore, and therefore a desired FRET efficiency.

In some embodiments, shield 302 comprises a polymer, such as a biomolecule or a dendritic polymer. FIG. 3C depicts examples of polymer shields and configurations of shielded recognition molecules of the disclosure. A first shielded construct 304 shows an example of a protein shield 330. In some embodiments, protein shield 330 forms a covalent linkage group between a recognition molecule and a label. For example, in some embodiments, protein shield 330 is attached to each of the recognition molecule and label through one or more covalent bonds, e.g., by covalent attachment through a side-chain of a natural or unnatural amino acid of protein shield 330. In some embodiments, an amino acid recognition molecule comprises a single polypeptide having at least one amino acid binding protein and protein shield 330 joined end-to-end.

Accordingly, in some aspects, the disclosure provides a shielded recognition molecule comprising a fusion polypeptide having an amino acid binding protein and a protein shield joined end-to-end (e.g., in a C-terminal to N-terminal fashion). In some embodiments, the binder and protein shield are joined end-to-end, either by a covalent bond or a linker that covalently joins the C-terminus of one protein to the N-terminus of the other protein. In some embodiments, a linker in the context of a fusion polypeptide refers to one or more amino acids within the fusion polypeptide that joins the binder and protein shield and that does not form part of the polypeptide sequence corresponding to either the binder or protein shield. In some embodiments, a linker comprises at least two amino acids (e.g., at least 2, 3, 4, 5, 6, 8, 10, 15, 25, 50, 100, or more, amino acids). In some embodiments, a linker comprises up to 5, up to 10, up to 15, up to 25, up to 50, or up to 100, amino acids. In some embodiments a linker comprises between about 2 and about 200 amino acids (e.g., between about 2 and about 100, between about 5 and about 50, between about 2 and about 20, between about 5 and about 20, or between about 2 and about 30, amino acids).

In some embodiments, a protein shield of a fusion polypeptide is a protein having a molecular weight of at least 10 kDa. For example, in some embodiments, a protein shield is a protein having a molecular weight of at least 10 kDa and up to 500 kDa (e.g., between about 10 kDa and about 250 kDa, between about 10 kDa and about 150 kDa, between about 10 kDa and about 100 kDa, between about 20 kDa and about 80 kDa, between about 15 kDa and about 100 kDa, or between about 15 kDa and about 50 kDa). In some embodiments, a protein shield of a fusion polypeptide is a protein comprising at least 25 amino acids. For example, in some embodiments, a protein shield is a protein comprising at least 25 and up to 1,000 amino acids (e.g., between about 100 and about 1,000 amino acids, between about 100 and about 750 amino acids, between about 500 and about 1,000 amino acids, between about 250 and about 750 amino acids, between about 50 and about 500 amino acids, between about 100 and about 400 amino acids, or between about 50 and about 250 amino acids).

In some embodiments, a protein shield is a polypeptide comprising one or more tag proteins. In some embodiments, a protein shield is a polypeptide comprising at least two tag proteins. In some embodiments, the at least two tag proteins are the same (e.g., the polypeptide comprises at least two copies of a tag protein sequence). In some embodiments, the at least two tag proteins are different (e.g., the polypeptide comprises at least two different tag protein sequences). Examples of tag proteins include, without limitation, Fasciola hepatica 8-kDa antigen (Fh8), Maltose-binding protein (MBP), N-utilization substance (NusA), Thioredoxin (Trx), Small ubiquitin-like modifier (SUMO), Glutathione-S-transferase (GST), Solubility-enhancer peptide sequences (SET), IgG domain B1 of Protein G (GB1), IgG repeat domain ZZ of Protein A (ZZ), Mutated dehalogenase (HaloTag), Solubility eNhancing Ubiquitous Tag (SNUT), Seventeen kilodalton protein (Skp), Phage T7 protein kinase (T7PK), E. coli secreted protein A (EspA), Monomeric bacteriophage T7 0.3 protein (Orc protein; Mocr), E. coli trypsin inhibitor (Ecotin), Calcium-binding protein (CaBP), Stress-responsive arsenate reductase (ArsC), N-terminal fragment of translation initiation factor IF2 (IF2-domain I), Stress-responsive proteins (e.g., RpoA, SlyD, Tsf, RpoS, PotD, Crr), and E. coli acidic proteins (e.g., msyB, yjgD, rpoD). See, e.g., Costa, S., et al. “Fusion tags for protein solubility, purification and immunogenicity in Escherichia coli: the novel Fh8 system.” Front Microbiol. 2014 Feb. 19; 5:63, the relevant content of which is incorporated herein by reference.

As described herein, a shielding element of the disclosure can advantageously absorb, deflect, or otherwise block radiative and/or non-radiative decay emitted by a label component of an amino acid recognition molecule. Thus, it should be appreciated that a suitable protein shield of a fusion polypeptide can be readily selected by those skilled in the art. For example, the inventors have demonstrated the use of a variety of types of protein shields in the context of a fusion polypeptide, including polypeptides having an amino acid binding protein fused to an enzyme (e.g., DNA polymerase, glutathione S-transferase), a transport protein (e.g., maltose-binding protein), a fluorescent protein (e.g., GFP), and a commercially available tag protein (e.g., SNAP-tag®). The inventors have further demonstrated the use of fusion polypeptides having multiple copies of a protein shield oriented in tandem.

Accordingly, in some embodiments, the disclosure provides a fusion polypeptide having one or more tandemly-oriented amino acid binding proteins fused to one or more tandemly-oriented protein shields. In some embodiments, where a fusion polypeptide comprises two or more tandemly-oriented binders and/or two or more tandemly-oriented shields, a terminal end of one of the two or more binders is joined end-to-end with a terminal end of one of the two or more shields. Fusion polypeptides having tandem copies of two or more binders are described elsewhere herein, and in some embodiments, such fusions can further comprise a protein shield joined end-to-end with one of the two or more binders.

In some embodiments, protein shield 330 forms a non-covalent linkage group between a recognition molecule and a label. For example, in some embodiments, protein shield 330 is a monomeric or multimeric protein comprising one or more ligand-binding sites. In some embodiments, a non-covalent linkage group is formed through one or more ligand moieties bound to the one or more ligand-binding sites. Additional examples of non-covalent linkages formed by protein shields are described elsewhere herein.

A second shielded construct 306 shows an example of a double-stranded nucleic acid shield comprising a first oligonucleotide strand 332 hybridized with a second oligonucleotide strand 334. As shown, in some embodiments, the double-stranded nucleic acid shield can comprise a recognition molecule attached to first oligonucleotide strand 332, and a label attached to second oligonucleotide strand 334. In this way, the double-stranded nucleic acid shield forms a non-covalent linkage group between the recognition molecule and the label through oligonucleotide strand hybridization. In some embodiments, a recognition molecule and a label can be attached to the same oligonucleotide strand, which can provide a single-stranded nucleic acid shield or a double-stranded nucleic acid shield through hybridization with another oligonucleotide strand. In some embodiments, strand hybridization can provide increased rigidity within a linkage group to further enhance separation between the recognition molecule and the label.

Where shielding element 302 comprises a nucleic acid, the separation distance between a label and a recognition molecule can be measured by the distance between attachment sites on the nucleic acid (e.g., direct attachment or indirect attachment, such as through one or more additional shield polymers). In some embodiments, the distance between attachment sites on a nucleic acid can be measured by the number of nucleotides within the nucleic acid that occur between the label and the recognition molecule. It should be understood that the number of nucleotides can refer to either the number of nucleotide bases in a single-stranded nucleic acid or the number of nucleotide base pairs in a double-stranded nucleic acid.

Accordingly, in some embodiments, the attachment site of a recognition molecule and the attachment site of a label can be separated by between 5 and 200 nucleotides (e.g., between 5 and 150 nucleotides, between 5 and 100 nucleotides, between 5 and 50 nucleotides, between 10 and 100 nucleotides). It should be appreciated that any position in a nucleic acid can serve as an attachment site for a recognition molecule, a label, or one or more additional polymer shields. In some embodiments, an attachment site can be at or approximately at the 5′ or 3′ end, or at an internal position along a strand of the nucleic acid.

The non-limiting configuration of second shielded construct 306 illustrates an example of a shield that forms a non-covalent linkage through strand hybridization. A further example of non-covalent linkage is illustrated by a third shielded construct 308 comprising an oligonucleotide shield 336. In some embodiments, oligonucleotide shield 336 is a nucleic acid aptamer that binds a recognition molecule to form a non-covalent linkage. In some embodiments, the recognition molecule is a nucleic acid aptamer, and oligonucleotide shield 336 comprises an oligonucleotide strand that hybridizes with the aptamer to form a non-covalent linkage.

A fourth shielded construct 310 shows an example of a dendritic polymer shield 338. As used herein, in some embodiments, a dendritic polymer refers generally to a polyol or a dendrimer. Polyols and dendrimers have been described in the art, and may include branched dendritic structures optimized for a particular configuration. In some embodiments, dendritic polymer shield 338 comprises polyethylene glycol, tetraethylene glycol, poly(amidoamine), poly(propyleneimine), poly(propyleneamine), carbosilane, poly(L-lysine), or a combination of one or more thereof.

A dendrimer, or dendron, is a repetitively branched molecule that is typically symmetric around the core and that may adopt a spherical three-dimensional morphology. See, e.g., Astruc et al. (2010) Chem. Rev. 110:1857. Incorporation of such structures into a shield of the disclosure can provide for a protective effect through the steric inhibition of contacts between a label and one or more biomolecules associated therewith (e.g., a recognition molecule and/or a polypeptide associated with the recognition molecule). Refinement of the chemical and physical properties of the dendrimer through variation in primary structure of the molecule, including potential functionalization of the dendrimer surface, allows the shielding effects to be adjusted as desired. Dendrimers may be synthesized by a variety of techniques using a wide range of materials and branching reactions, as is known in the art. Such synthetic variation allows the properties of the dendrimer to be customized as necessary.

FIG. 3D depicts further example configurations of shielded recognition molecules of the disclosure. A protein-nucleic acid construct 312 shows an example of a shield comprising more than one polymer in the form of a protein and a double-stranded nucleic acid. In some embodiments, the protein portion of the shield is attached to the nucleic acid portion of the shield through a covalent linkage. In some embodiments, the attachment is through a non-covalent linkage. For example, in some embodiments, the protein portion of the shield is a monovalent or multivalent protein that forms at least one non-covalent linkage through a ligand moiety attached to a ligand-binding site of the monovalent or multivalent protein. In some embodiments, the protein portion of the shield comprises an avidin protein.

In some embodiments, a shielded recognition molecule of the disclosure is an avidin-nucleic acid construct 314. In some embodiments, avidin-nucleic acid construct 314 includes a shield comprising an avidin protein 340 and a double-stranded nucleic acid. As described herein, avidin protein 340 may be used to form a non-covalent linkage between one or more amino acid recognition molecules and one or more labels, either directly or indirectly, such as through one or more additional shield polymers described herein.

Avidin proteins are biotin-binding proteins, generally having a biotin binding site at each of four subunits of the avidin protein. Avidin proteins include, for example, avidin, streptavidin, traptavidin, tamavidin, bradavidin, xenavidin, and homologs and variants thereof. In some cases, the monomeric, dimeric, or tetrameric form of the avidin protein can be used. In some embodiments, the avidin protein of an avidin protein complex is streptavidin in a tetrameric form (e.g., a homotetramer). In some embodiments, the biotin binding sites of an avidin protein provide attachment sites for one or more amino acid recognition molecules, one or more labels, and/or one or more additional shield polymers described herein.

An illustrative diagram of an avidin protein complex is shown in the inset panel of FIG. 3D. As shown in the inset panel, avidin protein 340 can include a binding site 342 at each of four subunits of the protein which can be bound to a biotin moiety (shown as white circles). The multivalency of avidin protein 340 can allow for various linkage configurations, which are generally shown for illustrative purposes. For example, in some embodiments, a biotin linkage moiety 344 can be used to provide a single point of attachment to avidin protein 340. In some embodiments, a bis-biotin linkage moiety 346 can be used to provide two points of attachment to avidin protein 340. As illustrated by avidin-nucleic acid construct 314, an avidin protein complex may be formed by two bis-biotin linkage moieties, which form a trans-configuration to provide an increased separation distance between a recognition molecule and a label.

Various further examples of avidin protein shield configurations are shown. A first avidin construct 316 shows an example of an avidin shield attached to a recognition molecule through a bis-biotin linkage moiety and to two labels through separate biotin linkage moieties. A second avidin construct 318 shows an example of an avidin shield attached to two recognition molecules through separate biotin linkage moieties and to a label through a bis-biotin linkage moiety. A third avidin construct 320 shows an example of an avidin shield attached to two recognition molecules through separate biotin linkage moieties and to a labeled nucleic acid through a biotin linkage moiety of each strand of the nucleic acid. A fourth avidin construct 322 shows an example of an avidin shield attached to a recognition molecule and to a labeled nucleic acid through separate bis-biotin linkage moieties. As shown, the label is further shielded from the recognition molecule by a dendritic polymer between the label and nucleic acid. A fifth avidin construct 324 shows an example of an internal label 326 attached to two avidin-shielded recognition molecules. As shown, each recognition molecule is attached to a different avidin protein through a bis-biotin linkage moiety, and internal label 326 is attached to both avidin proteins through separate bis-biotin linkage moieties.

It should be appreciated that the example configurations of shielded recognition molecules shown in FIGS. 3A-3D are provided for illustrative purposes. The inventors have conceived of various other shield configurations using one or more different polymers that form a covalent or non-covalent linkage between recognition and label components of a shielded recognition molecule. By way of example, FIG. 3E illustrates the modularity of shield configurations in accordance with the disclosure.

As shown at the top of FIG. 3E, a shielded recognition molecule generally comprises a recognition component 350, a shielding element 352, and a label component 354. For ease of illustration, recognition component 350 is depicted as one amino acid recognition molecule, and label component 354 is depicted as one label.

It should be appreciated that shielded recognition molecules of the disclosure can comprise shielding element 352 attached to one or more amino acid recognition molecules and one or more labels. Where recognition component 350 comprises more than one recognition molecule, each recognition molecule can be attached to shielding element 352 at one or more attachment sites on shielding element 352. In some embodiments, recognition component 350 comprises a single polypeptide fusion construct having tandem copies of two or more amino acid binding proteins, as described elsewhere herein. Where label component 354 comprises more than one label, each label can be attached to shielding element 352 at one or more attachment sites on shielding element 352. While label component 354 is generically shown as having a single attachment point, it is not limited in this respect. For example, in some embodiments, an internal label having more than one attachment point can be used to join more than one recognition component 350 and/or shielding element 352, as illustrated by avidin construct 324.

In some embodiments, shielding element 352 comprises a protein 360. In some embodiments, protein 360 is a monovalent or multivalent protein. In some embodiments, protein 360 is a monomeric or multimeric protein, such as a protein homodimer, protein heterodimer, protein oligomer, or other proteinaceous molecule. In some embodiments, shielding element 352 comprises a protein complex formed by a protein non-covalently bound to at least one other molecule. For example, in some embodiments, shielding element 352 comprises a protein-protein complex 362. In some embodiments, protein-protein complex 362 comprises one proteinaceous molecule specifically bound to another proteinaceous molecule. In some embodiments, protein-protein complex 362 comprises an antibody or antibody fragment (e.g., scFv) bound to an antigen. In some embodiments, protein-protein complex 362 comprises a receptor bound to a protein ligand. Additional examples of protein-protein complexes include, without limitation, trypsin-aprotinin, barnase-barstar, and colicin E9-Im9 immunity protein.

In some embodiments, shielding element 352 comprises a protein-ligand complex 364. In some embodiments, protein-ligand complex 364 comprises a monovalent protein and a non-proteinaceous ligand moiety. For example, in some embodiments, protein-ligand complex 364 comprises an enzyme bound to a small-molecule inhibitor moiety. In some embodiments, protein-ligand complex 364 comprises a receptor bound to a non-proteinaceous ligand moiety.

In some embodiments, shielding element 352 comprises a multivalent protein complex formed by a multivalent protein non-covalently bound to one or more ligand moieties. In some embodiments, shielding element 352 comprises an avidin protein complex formed by an avidin protein non-covalently bound to one or more biotin linkage moieties. Constructs 366, 368, 370, and 372 provide illustrative examples of avidin protein complexes, any one or more of which may be incorporated into shielding element 352.

In some embodiments, shielding element 352 comprises a two-way avidin complex 366 comprising an avidin protein bound to two bis-biotin linkage moieties. In some embodiments, shielding element 352 comprises a three-way avidin complex 368 comprising an avidin protein bound to two biotin linkage moieties and a bis-biotin linkage moiety. In some embodiments, shielding element 352 comprises a four-way avidin complex 370 comprising an avidin protein bound to four biotin linkage moieties.

In some embodiments, shielding element 352 comprises an avidin protein comprising one or two non-functional binding sites engineered into the avidin protein. For example, in some embodiments, shielding element 352 comprises a divalent avidin complex 372 comprising an avidin protein bound to a biotin linkage moiety at each of two subunits, where the avidin protein comprises a non-functional ligand-binding site 348 at each of two other subunits. As shown, in some embodiments, divalent avidin complex 372 comprises a trans-divalent avidin protein, although a cis-divalent avidin protein may be used depending on a desired implementation. In some embodiments, the avidin protein is a trivalent avidin protein. In some embodiments, the trivalent avidin protein comprises non-functional ligand-binding site 348 at one subunit and is bound to three biotin linkage moieties, or one biotin linkage moiety and one bis-biotin linkage moiety, at the other subunits.

In some embodiments, shielding element 352 comprises a dendritic polymer 374. In some embodiments, dendritic polymer 374 is a polyol or a dendrimer, as described elsewhere herein. In some embodiments, dendritic polymer 374 is a branched polyol or a branched dendrimer. In some embodiments, dendritic polymer 374 comprises a monosaccharide-TEG, a disaccharide, an N-acetyl monosaccharide, a TEMPO-TEG, a trolox-TEG, or a glycerol dendrimer. Examples of polyols useful in accordance with shielded recognition molecules of the disclosure include polyether polyols and polyester polyols, e.g., polyethylene glycol, polypropylene glycol, and similar such polymers well known in the art. In some embodiments, dendritic polymer 374 comprises a compound of the following formula: —(CH₂CH₂O)ₙ—, where n is an integer from 1 to 500, inclusive. In some embodiments, dendritic polymer 374 comprises a compound of the following formula: —(CH₂CH₂O)ₙ—, wherein n is an integer from 1 to 100, inclusive.

In some embodiments, shielding element 352 comprises a nucleic acid. In some embodiments, the nucleic acid is single-stranded. In some embodiments, label component 354 is attached directly or indirectly to one end of the single-stranded nucleic acid (e.g., the 5′ end or the 3′ end) and recognition component 350 is attached directly or indirectly to the other end of the single-stranded nucleic acid (e.g., the 3′ end or the 5′ end). For example, the single-stranded nucleic acid can comprise a label attached to the 5′ end of the nucleic acid and an amino acid recognition molecule attached to the 3′ end of the nucleic acid.

In some embodiments, shielding element 352 comprises a double-stranded nucleic acid 376. As shown, in some embodiments, double-stranded nucleic acid 376 can form a non-covalent linkage between recognition component 350 and label component 354 through strand hybridization. However, in some embodiments, double-stranded nucleic acid 376 can form a covalent linkage between recognition component 350 and label component 354 through attachment to the same oligonucleotide strand. In some embodiments, label component 354 is attached directly or indirectly to one end of the double-stranded nucleic acid and recognition component 350 is attached directly or indirectly to the other end of the double-stranded nucleic acid. For example, the double-stranded nucleic acid can comprise a label attached to the 5′ end of one strand and an amino acid recognition molecule attached to the 5′ end of the other strand.

In some embodiments, shielding element 352 comprises a nucleic acid that forms one or more structural motifs which can be useful for increasing steric bulk of the shield. Examples of nucleic acid structural motifs include, without limitation, stem-loops, three-way junctions (e.g., formed by two or more stem-loop motifs), four-way junctions (e.g., Holliday junctions), and bulge loops.

In some embodiments, shielding element 352 comprises a nucleic acid that forms a stem-loop 378. A stem-loop, or hairpin loop, is an unpaired loop of nucleotides on an oligonucleotide strand that is formed when the oligonucleotide strand folds and forms base pairs with another section of the same strand. In some embodiments, the unpaired loop of stem-loop 378 comprises three to ten nucleotides. Accordingly, stem-loop 378 can be formed by two regions of an oligonucleotide strand having inverted complementary sequences that hybridize to form a stem, where the two regions are separated by the three to ten nucleotides that form the unpaired loop. In some embodiments, the stem of stem-loop 378 can be designed to have one or more G/C nucleotides, which can provide added stability with the addition hydrogen bonding interaction that forms compared to A/T nucleotides. In some embodiments, the stem of stem-loop 378 comprises G/C nucleotides immediately adjacent to an unpaired loop sequence. In some embodiments, the stem of stem-loop 378 comprises G/C nucleotides within the first 2, 3, 4, or 5 nucleotides adjacent to an unpaired loop sequence. In some embodiments, an unpaired loop of stem-loop 378 comprises one or more attachment sites. In some embodiments, an attachment site occurs at an abasic site in the unpaired loop. In some embodiments, an attachment site occurs at a base of the unpaired loop.

In some embodiments, stem-loop 378 is formed by a double-stranded nucleic acid. As described herein, in some embodiments, the double-stranded nucleic acid can form a non-covalent linkage group through strand hybridization of first and second oligonucleotide strands. However, in some embodiments, shielding element 352 comprises a single-stranded nucleic acid that forms a stem-loop motif, e.g., to provide a covalent linkage group. In some embodiments, shielding element 352 comprises a nucleic acid that forms two or more stem-loop motifs. For example, in some embodiments, the nucleic acid comprises two stem-loop motifs. In some embodiments, a stem of one stem-loop motif is adjacent to the stem of the other such that the motifs together form a three-way junction. In some embodiments, shielding element 352 comprises a nucleic acid that forms a four-way junction 380. In some embodiments, four-way junction 380 is formed through hybridization of two or more oligonucleotide strands (e.g., 2, 3, or 4 oligonucleotide strands).

In some embodiments, shielding element 352 comprises one or more polymers selected from 360, 362, 364, 366, 368, 370, 372, 374, 376, 378, and 380 of FIG. 3E. It should be appreciated that the linkage moieties and attachment sites shown on each of 360, 362, 364, 366, 368, 370, 372, 374, 376, 378, and 380 are shown for illustrative purposes and are not intended to depict a preferred linkage or attachment site configuration.

In some aspects, the disclosure provides an amino acid recognition molecule of Formula (II):

A-(Y)ₙ-D (II),

wherein: A is an amino acid binding component comprising at least one amino acid recognition molecule; each instance of Y is a polymer that forms a covalent or non-covalent linkage group; n is an integer from 1 to 10, inclusive; and D is a label component comprising at least one detectable label. In some embodiments, the disclosure provides a composition comprising a soluble amino acid recognition molecule of Formula (II).

In some embodiments, A comprises a plurality of amino acid recognition molecules. In some embodiments, each amino acid recognition molecule of the plurality is attached to a different attachment site on Y. In some embodiments, at least two amino acid recognition molecules of the plurality are attached to a single attachment site on Y. In some embodiments, the amino acid recognition molecule is a recognition protein or a nucleic acid aptamer, e.g., as described elsewhere herein.

In some embodiments, the detectable label is a luminescent label or a conductivity label. In some embodiments, the luminescent label comprises at least one fluorophore dye molecule. In some embodiments, D comprises 20 or fewer fluorophore dye molecules. In some embodiments, the ratio of the number of fluorophore dye molecules to the number of amino acid recognition molecules is between 1:1 and 20:1. In some embodiments, the luminescent label comprises at least one FRET pair comprising a donor label and an acceptor label. In some embodiments, the ratio of the donor label to the acceptor label is 1:1, 2:1, 3:1, 4:1, or 5:1. In some embodiments, the ratio of the acceptor label to the donor label is 1:1, 2:1, 3:1, 4:1, or 5:1.

In some embodiments, D is less than 200 Å in diameter. In some embodiments, —(Y)ₙ— is at least 2 nm in length. In some embodiments, —(Y)ₙ— is at least 5 nm in length. In some embodiments, —(Y)ₙ— is at least 10 nm in length. In some embodiments, each instance of Y is independently a biomolecule, a polyol, or a dendrimer. In some embodiments, the biomolecule is a nucleic acid, a polypeptide, or a polysaccharide.

In some embodiments, the amino acid recognition molecule is of one of the following formulae:

A-Y¹—(Y)ₘ-D or A-(Y)ₘ—Y¹-D,

wherein: Y¹ is a nucleic acid or a polypeptide; and m is an integer from 0 to 10, inclusive.

In some embodiments, the nucleic acid comprises a first oligonucleotide strand. In some embodiments, the nucleic acid comprises a second oligonucleotide strand hybridized with the first oligonucleotide strand. In some embodiments, the nucleic acid forms a covalent linkage through the first oligonucleotide strand. In some embodiments, the nucleic acid forms a non-covalent linkage through the hybridized first and second oligonucleotide strands.

In some embodiments, the polypeptide is a monovalent or multivalent protein. In some embodiments, the monovalent or multivalent protein forms at least one non-covalent linkage through a ligand moiety attached to a ligand-binding site of the monovalent or multivalent protein. In some embodiments, A, Y, or D comprises the ligand moiety.

In some embodiments, the amino acid recognition molecule is of one of the following formulae:

A-(Y)ₘ—Y²-D or A-Y²—(Y)ₘ-D,

wherein: Y² is a polyol or dendrimer; and m is an integer from 0 to 10, inclusive. In some embodiments, the polyol or dendrimer comprises polyethylene glycol, tetraethylene glycol, poly(amidoamine), poly(propyleneimine), poly(propyleneamine), carbosilane, poly(L-lysine), or a combination of one or more thereof.

In some aspects, the disclosure provides an amino acid recognition molecule of Formula (III):

A-Y¹-D (III),

wherein: A is an amino acid binding component comprising at least one amino acid recognition molecule; Y¹ is a nucleic acid or a polypeptide; D is a label component comprising at least one detectable label. In some embodiments, when Y¹ is a nucleic acid, the nucleic acid forms a covalent or non-covalent linkage group. In some embodiments, when Y¹ is a polypeptide, the polypeptide forms a non-covalent linkage group characterized by a dissociation constant (K_{D}) of less than 50×10⁻⁹ M.

In some embodiments, Y¹ is a nucleic acid comprising a first oligonucleotide strand. In some embodiments, the nucleic acid comprises a second oligonucleotide strand hybridized with the first oligonucleotide strand. In some embodiments, A is attached to the first oligonucleotide strand, and wherein D is attached to the second oligonucleotide strand. In some embodiments, A is attached to a first attachment site on the first oligonucleotide strand, and wherein D is attached to a second attachment site on the first oligonucleotide strand. In some embodiments, each oligonucleotide strand of the nucleic acid comprises fewer than 150, fewer than 100, or fewer than 50 nucleotides.

In some embodiments, Y¹ is a monovalent or multivalent protein. In some embodiments, the monovalent or multivalent protein forms at least one non-covalent linkage through a ligand moiety attached to a ligand-binding site of the monovalent or multivalent protein. In some embodiments, at least one of A and D comprises the ligand moiety. In some embodiments, the polypeptide is an avidin protein (e.g., avidin, streptavidin, traptavidin, tamavidin, bradavidin, xenavidin, or a homolog or variant thereof). In some embodiments, the ligand moiety is a biotin moiety.

In some embodiments, the amino acid recognition molecule is of one of the following formulae:

A-Y¹-(Y)ₙ-D or A-(Y)ₙ—Y¹-D,

wherein: each instance of Y is a polymer that forms a covalent or non-covalent linkage group; and n is an integer from 1 to 10, inclusive. In some embodiments, each instance of Y is independently a biomolecule, a polyol, or a dendrimer.

In other aspects, the disclosure provides an amino acid recognition molecule comprising: a nucleic acid; at least one amino acid recognition molecule attached to a first attachment site on the nucleic acid; and at least one detectable label attached to a second attachment site on the nucleic acid. In some embodiments, the nucleic acid forms a covalent or non-covalent linkage group between the at least one amino acid recognition molecule and the at least one detectable label.

In some embodiments, the nucleic acid is a double-stranded nucleic acid comprising a first oligonucleotide strand hybridized with a second oligonucleotide strand. In some embodiments, the first attachment site is on the first oligonucleotide strand, and wherein the second attachment site is on the second oligonucleotide strand. In some embodiments, the at least one amino acid recognition molecule is attached to the first attachment site through a protein that forms a covalent or non-covalent linkage group between the at least one amino acid recognition molecule and the nucleic acid. In some embodiments, the at least one detectable label is attached to the second attachment site through a protein that forms a covalent or non-covalent linkage group between the at least one detectable label and the nucleic acid. In some embodiments, the first and second attachment sites are separated by between 5 and 100 nucleotide bases or nucleotide base pairs on the nucleic acid.

In yet other aspects, the disclosure provides an amino acid recognition molecule comprising: a multivalent protein comprising at least two ligand-binding sites; at least one amino acid recognition molecule attached to the protein through a first ligand moiety bound to a first ligand-binding site on the protein; and at least one detectable label attached to the protein through a second ligand moiety bound to a second ligand-binding site on the protein.

In some embodiments, the multivalent protein is an avidin protein comprising four ligand-binding sites. In some embodiments, the ligand-binding sites are biotin binding sites, and the ligand moieties are biotin moieties. In some embodiments, at least one of the biotin moieties is a bis-biotin moiety, and the bis-biotin moiety is bound to two biotin binding sites on the avidin protein. In some embodiments, the at least one amino acid recognition molecule is attached to the protein through a nucleic acid comprising the first ligand moiety. In some embodiments, the at least one detectable label is attached to the protein through a nucleic acid comprising the second ligand moiety.

In some aspects, the disclosure provides labeled reagents comprising a shielding element that protects a target molecule from label-induced photodamage. In some embodiments, a labeled reagent has a structure of Formula (IVa):

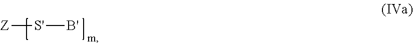

wherein: Z is a multivalent central core element comprising a luminescent label; each S′ is independently an intermediate chemical group, wherein at least one S′ comprises a shielding element; each B′ is independently a terminal chemical group, wherein at least one B′ comprises a binding element that binds a target molecule; and m is an integer from 2 to 24, inclusive.

In some embodiments, Z comprises a multivalent fluorescent dye element. In some embodiments, Z comprises a multivalent cyanine dye. In some embodiments, Z comprises a luminescent label other than a fluorescent dye. In some embodiments, Z comprises a FRET label (e.g., one or more chromophores of a FRET pair).

In some embodiments, m is an integer from 2 to 12, inclusive. In some embodiments, m is an integer from 2 to 8, inclusive. In some embodiments, m is an integer from 2 to 4, inclusive.

In some embodiments, a labeled reagent has a structure of Formula (IVb), (IVc), or (IVd):

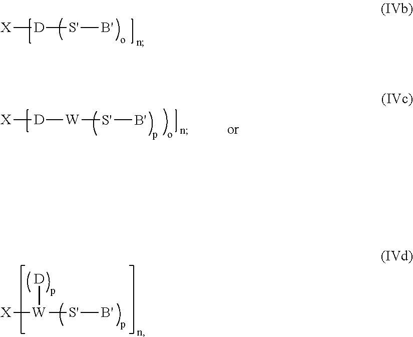

wherein: X is a non-luminescent multivalent central core element; each instance of D is independently a luminescent label or a covalent bond, with the proviso that at least one instance of D is a luminescent label; each instance of W, if present, is a branching element; each S′ is independently an intermediate chemical group, wherein at least one S′ comprises a shielding element; each B′ is independently a terminal chemical group, wherein at least one B′ comprises a binding element that binds a target molecule; each instance of n is independently an integer from 2 to 6, inclusive; each instance of o is independently an integer from 1 to 4, inclusive; and each instance of p is independently an integer from 1 to 4, inclusive.

In some embodiments, X comprises a polyamine. In some embodiments, X comprises a tertiary amide. In some embodiments, X comprises a substituted triazine group (e.g., a trisubstituted triazine). In some embodiments, X comprises a substituted phenyl group (e.g., a disubstituted or trisubstituted phenyl). In some embodiments, X comprises a substituted carbocyclic group (e.g., a substituted cyclohexane). In some embodiments, X comprises a secondary, tertiary, or quaternary carbon atom.

In some embodiments, D comprises a fluorescent dye. In some embodiments, D comprises a FRET label (e.g., one or more chromophores of a FRET pair).

In some embodiments, W comprises the structure:

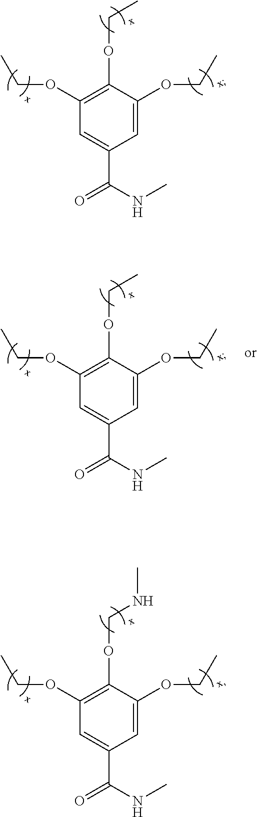

wherein each instance of x is independently an integer from 1 to 6, inclusive. In some embodiments, each instance of x is independently an integer from 1 to 4, inclusive.

Referring to Formulae (IVa)-(IVd) above, in some embodiments, the shielding element decreases photodamage of the binding element and/or of a target molecule associated with the binding element. In some embodiments, the shielding element decreases contact between the luminescent label and the binding element. In some embodiments, the shielding element decreases contact between the luminescent label and a target molecule associated with the binding element.

In some embodiments, the binding element comprises a biotin moiety. In some embodiments, the binding element comprises an amino acid recognition molecule (e.g., an amino acid binding protein). For example, in some embodiments, the binding element comprises an amino acid recognition molecule, and the target molecule comprises a polypeptide.

In some embodiments, the shielding element comprises a plurality of side chains. In some embodiments, at least one side chain has a molecular weight of at least 300 g/mol (e.g., at least 350, at least 400, at least 450, or at least 500 g/mol). In some embodiments, at least one side chain has a molecular weight of between about 300 and 1,000 g/mol (e.g., 350-1,000, 400-1,000, 450-1,000, or 500-1,000 g/mol). In some embodiments, all of the side chains have a molecular weight of at least 300 g/mol.

In some embodiments, the shielding element comprises at least one side chain comprising a dendrimer, a polyethylene glycol, or a negatively-charged component. In some embodiments, the negatively-charged component comprises a sulfonic acid. In some embodiments, the shielding element comprises at least one side chain comprising a substituted phenyl group. In some embodiments, the at least one side chain comprises the structure:

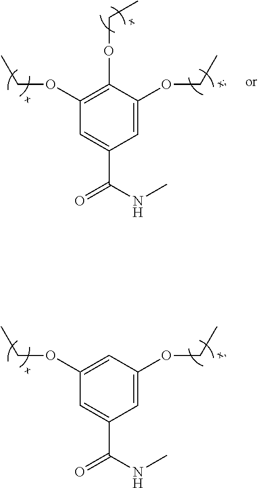

wherein each instance of x is independently an integer from 1 to 6, inclusive. In some embodiments, each instance of x is independently an integer from 1 to 4, inclusive.

In some embodiments, the shielding element comprises the structure:

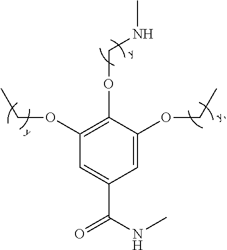

wherein each instance of y is independently an integer from 1 to 6, inclusive.

As described elsewhere herein, shielded recognition molecules of the disclosure may be used in a polypeptide sequencing method in accordance with the disclosure, or any method known in the art. For example, in some embodiments, a shielded recognition molecule provided herein may be used in an Edman-type degradation reaction provided herein, or conventionally known in the art, which can involve iterative cycling of multiple reaction mixtures in a polypeptide sequencing reaction. In some embodiments, a shielded recognition molecule provided herein may be used in a dynamic sequencing reaction of the disclosure, which involves amino acid recognition and degradation in a single reaction mixture.

### Cleaving Reagents

In some embodiments, a cleaving reagent of the disclosure is an exopeptidase. An exopeptidase generally requires a polypeptide substrate to comprise at least one of a free amino group at its amino-terminus or a free carboxyl group at its carboxy-terminus. In some embodiments, an exopeptidase in accordance with the disclosure hydrolyses a bond at or near a terminus of a polypeptide. In some embodiments, an exopeptidase hydrolyses a bond not more than three residues from a polypeptide terminus. For example, in some embodiments, a single hydrolysis reaction catalyzed by an exopeptidase cleaves a single amino acid, a dipeptide, or a tripeptide from a polypeptide terminal end.

In some embodiments, an exopeptidase in accordance with the disclosure is an aminopeptidase or a carboxypeptidase, which cleaves a single amino acid from an amino- or a carboxy-terminus, respectively. In some embodiments, an exopeptidase in accordance with the disclosure is a dipeptidyl-peptidase or a peptidyl-dipeptidase, which cleave a dipeptide from an amino- or a carboxy-terminus, respectively. In yet other embodiments, an exopeptidase in accordance with the disclosure is a tripeptidyl-peptidase, which cleaves a tripeptide from an amino-terminus. Peptidase classification and activities of each class or subclass thereof is well known and described in the literature (see, e.g., Gurupriya, V. S. & Roy, S. C. Proteases and Protease Inhibitors in Male Reproduction. Proteases in Physiology and Pathology 195-216 (2017); and Brix, K. & Stöcker, W. Proteases: Structure and Function. Chapter 1). In some embodiments, a peptidase in accordance with the disclosure removes more than three amino acids from a polypeptide terminus. Accordingly, in some embodiments, the peptidase is an endopeptidase, e.g., that cleaves preferentially at particular positions (e.g., before or after a particular amino acid). In some embodiments, the size of a polypeptide cleavage product of endopeptidase activity will depend on the distribution of cleavage sites (e.g., amino acids) within the polypeptide being analyzed.

An exopeptidase in accordance with the disclosure may be selected or engineered based on the directionality of a sequencing reaction. For example, in embodiments of sequencing from an amino-terminus to a carboxy-terminus of a polypeptide, an exopeptidase comprises aminopeptidase activity. Conversely, in embodiments of sequencing from a carboxy-terminus to an amino-terminus of a polypeptide, an exopeptidase comprises carboxypeptidase activity. Examples of carboxypeptidases that recognize specific carboxy-terminal amino acids, which may be used as labeled exopeptidases or inactivated to be used as non-cleaving labeled recognition molecules described herein, have been described in the literature (see, e.g., Garcia-Guerrero, M. C., et al. (2018) PNAS 115(17)).

Suitable peptidases for use as cleaving reagents and/or recognition molecules include aminopeptidases that selectively bind one or more types of amino acids. In some embodiments, an aminopeptidase recognition molecule is modified to inactivate aminopeptidase activity. In some embodiments, an aminopeptidase cleaving reagent is non-specific such that it cleaves most or all types of amino acids from a terminal end of a polypeptide. In some embodiments, an aminopeptidase cleaving reagent is more efficient at cleaving one or more types of amino acids from a terminal end of a polypeptide as compared to other types of amino acids at the terminal end of the polypeptide. For example, an aminopeptidase in accordance with the disclosure specifically cleaves alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and/or valine. In some embodiments, an aminopeptidase is a proline aminopeptidase. In some embodiments, an aminopeptidase is a proline iminopeptidase. In some embodiments, an aminopeptidase is a glutamate/aspartate-specific aminopeptidase. In some embodiments, an aminopeptidase is a methionine-specific aminopeptidase.

In some embodiments, an aminopeptidase is a non-specific aminopeptidase. In some embodiments, a non-specific aminopeptidase is a zinc metalloprotease.

Examples of cleaving reagents (e.g., aminopeptidases) for use in accordance with the disclosure are described more fully in PCT International Application No. PCT/US2019/061831, filed Nov. 15, 2019, and PCT International Application No. PCT/US2021/033493, filed May 20, 2021, the relevant content of which is incorporated herein by reference in its entirety.

### Luminescent Labels

As used herein, a luminescent label is a molecule that absorbs one or more photons and may subsequently emit one or more photons after one or more time durations. In some embodiments, the term is used interchangeably with “label” or “luminescent molecule” depending on context. A luminescent label in accordance with certain embodiments described herein may refer to a luminescent label of a labeled recognition molecule, a luminescent label of a labeled peptidase (e.g., a labeled exopeptidase, a labeled non-specific exopeptidase), a luminescent label of a labeled peptide, a luminescent label of a labeled cofactor, or another labeled composition described herein. In some embodiments, a luminescent label in accordance with the disclosure refers to a labeled amino acid of a labeled polypeptide comprising one or more labeled amino acids.

In some embodiments, a luminescent label may comprise a first and second chromophore. In some embodiments, an excited state of the first chromophore is capable of relaxation via an energy transfer to the second chromophore. In some embodiments, the energy transfer is a Förster resonance energy transfer (FRET). Such a FRET pair may be useful for providing a luminescent label with properties that make the label easier to differentiate from amongst a plurality of luminescent labels in a mixture. In yet other embodiments, a FRET pair comprises a first chromophore of a first luminescent label and a second chromophore of a second luminescent label. In certain embodiments, the FRET pair may absorb excitation energy in a first spectral range and emit luminescence in a second spectral range. In general, a donor chromophore is selected that has a substantial spectrum of the acceptor chromophore. Furthermore, it may also be desirable in certain applications that the donor have an excitation maximum near a laser frequency such as Helium-Cadmium 442 nM, Argon 488 nM, NdrYAG 532 nm, He—Ne 633 nm, etc. In such applications, the use of intense laser light can serve as an effective means to excite the donor fluorophore.

In some embodiments, an acceptor chromophore of a FRET label has a substantial overlap of its excitation spectrum with the emission spectrum of a donor chromophore of the FRET label. In some embodiments, the wavelength maximum of the emission spectrum of the acceptor chromophore is preferably at least 10 nm greater than the wavelength maximum of the excitation spectrum of the donor chromophore. Additional examples of useful FRET labels include, e.g., those described in U.S. Pat. Nos. 5,654,419, 5,688,648, 5,853,992, 5,863,727, 5,945,526, 6,008,373, 6,150,107, 6,177,249, 6,335,440, 6,348, 596, 6,479,303, 6,545,164, 6,849,745, 6,696,255, and 6,908,769 and Published U.S. Patent Application Nos. 2002/0168641, 2003/0143594. and 2004/0076979, the disclosures of which are incorporated herein by reference for all purposes.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluoroscein, rhodamine, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho 101, ATTO Rho11, ATTO Rho 12, ATTO Rho 13, ATTO Rho 14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Co1, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-RO, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-05, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Sera™ 632, Sera™ 633, Sera™ 650, Sera™ 660, Sera™ 670, Sera™ 680, Sera™ 700, Sera™ 750, Sera™ 780, Sera™ APC-780, Sera™ PerCP-680, Sera™ R-PE-670, Sera™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

### Luminescence

In some aspects, the disclosure relates to polypeptide sequencing and/or identification based on one or more luminescence properties of a luminescent label. In some embodiments, a luminescent label is identified based on luminescence lifetime, luminescence intensity, brightness, absorption spectra, emission spectra, luminescence quantum yield, or a combination of two or more thereof. In some embodiments, a plurality of types of luminescent labels can be distinguished from each other based on different luminescence lifetimes, luminescence intensities, brightnesses, absorption spectra, emission spectra, luminescence quantum yields, or combinations of two or more thereof. In some embodiments, a luminescent label is identified based on luminescence intensity alone. Identifying may mean assigning the exact identity and/or quantity of one type of amino acid (e.g., a single type or a subset of types) associated with a luminescent label, and may also mean assigning an amino acid location in a polypeptide relative to other types of amino acids.

In some embodiments, luminescence is detected by exposing a luminescent label to a series of separate light pulses and evaluating the timing or other properties of each photon that is emitted from the label. In some embodiments, information for a plurality of photons emitted sequentially from a label is aggregated and evaluated to identify the label and thereby identify an associated type of amino acid. In some embodiments, a luminescence lifetime of a label is determined from a plurality of photons that are emitted sequentially from the label, and the luminescence lifetime can be used to identify the label. In some embodiments, a luminescence intensity of a label is determined from a plurality of photons that are emitted sequentially from the label, and the luminescence intensity can be used to identify the label. In some embodiments, a luminescence lifetime and luminescence intensity of a label is determined from a plurality of photons that are emitted sequentially from the label, and the luminescence lifetime and luminescence intensity can be used to identify the label.

In some aspects of the disclosure, a single polypeptide molecule is exposed to a plurality of separate light pulses and a series of emitted photons are detected and analyzed. In some embodiments, the series of emitted photons provides information about the single polypeptide molecule that is present and that does not change in the reaction sample over the time of the experiment. However, in some embodiments, the series of emitted photons provides information about a series of different molecules that are present at different times in the reaction sample (e.g., as a reaction or process progresses). By way of example and not limitation, such information may be used to sequence and/or identify a polypeptide subjected to chemical or enzymatic degradation in accordance with the disclosure.

In certain embodiments, a luminescent label absorbs one photon and emits one photon after a time duration. In some embodiments, the luminescence lifetime of a label can be determined or estimated by measuring the time duration. In some embodiments, the luminescence lifetime of a label can be determined or estimated by measuring a plurality of time durations for multiple pulse events and emission events. In some embodiments, the luminescence lifetime of a label can be differentiated amongst the luminescence lifetimes of a plurality of types of labels by measuring the time duration. In some embodiments, the luminescence lifetime of a label can be differentiated amongst the luminescence lifetimes of a plurality of types of labels by measuring a plurality of time durations for multiple pulse events and emission events. In certain embodiments, a label is identified or differentiated amongst a plurality of types of labels by determining or estimating the luminescence lifetime of the label. In certain embodiments, a label is identified or differentiated amongst a plurality of types of labels by differentiating the luminescence lifetime of the label amongst a plurality of the luminescence lifetimes of a plurality of types of labels.

Determination of a luminescence lifetime of a luminescent label can be performed using any suitable method (e.g., by measuring the lifetime using a suitable technique or by determining time-dependent characteristics of emission). In some embodiments, determining the luminescence lifetime of one label comprises determining the lifetime relative to another label. In some embodiments, determining the luminescence lifetime of a label comprises determining the lifetime relative to a reference. In some embodiments, determining the luminescence lifetime of a label comprises measuring the lifetime (e.g., fluorescence lifetime). In some embodiments, determining the luminescence lifetime of a label comprises determining one or more temporal characteristics that are indicative of lifetime. In some embodiments, the luminescence lifetime of a label can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescence lifetime of a label can be distinguished from a plurality of labels having different luminescence lifetimes based on the distribution of photon arrival times measured with respect to an excitation pulse.

It should be appreciated that a luminescence lifetime of a luminescent label is indicative of the timing of photons emitted after the label reaches an excited state and the label can be distinguished by information indicative of the timing of the photons. Some embodiments may include distinguishing a label from a plurality of labels based on the luminescence lifetime of the label by measuring times associated with photons emitted by the label. The distribution of times may provide an indication of the luminescence lifetime which may be determined from the distribution. In some embodiments, the label is distinguishable from the plurality of labels based on the distribution of times, such as by comparing the distribution of times to a reference distribution corresponding to a known label. In some embodiments, a value for the luminescence lifetime is determined from the distribution of times.

As used herein, in some embodiments, luminescence intensity refers to the number of emitted photons per unit time that are emitted by a luminescent label which is being excited by delivery of a pulsed excitation energy. In some embodiments, the luminescence intensity refers to the detected number of emitted photons per unit time that are emitted by a label which is being excited by delivery of a pulsed excitation energy, and are detected by a particular sensor or set of sensors. In some embodiments, the luminescence intensity of a label can be differentiated amongst the luminescence intensities of a plurality of types of labels (e.g., FRET labels). In some embodiments, a label is identified or differentiated amongst a plurality of types of labels by determining or estimating the luminescence intensity of the label. In some embodiments, a label is identified or differentiated amongst a plurality of types of labels by differentiating the luminescence intensity of the label amongst a plurality of the luminescence intensities of a plurality of types of labels.

As used herein, in some embodiments, brightness refers to a parameter that reports on the average emission intensity per luminescent label. Thus, in some embodiments, “emission intensity” may be used to generally refer to brightness of a composition comprising one or more labels. In some embodiments, brightness of a label is equal to the product of its quantum yield and extinction coefficient.

As used herein, in some embodiments, luminescence quantum yield refers to the fraction of excitation events at a given wavelength or within a given spectral range that lead to an emission event, and is typically less than 1. In some embodiments, the luminescence quantum yield of a luminescent label described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a label is identified by determining or estimating the luminescence quantum yield.

As used herein, in some embodiments, an excitation energy is a pulse of light from a light source. In some embodiments, an excitation energy is in the visible spectrum. In some embodiments, an excitation energy is in the ultraviolet spectrum. In some embodiments, an excitation energy is in the infrared spectrum. In some embodiments, an excitation energy is at or near the absorption maximum of a luminescent label from which a plurality of emitted photons are to be detected. In certain embodiments, the excitation energy is between about 500 nm and about 700 nm (e.g., between about 500 nm and about 600 nm, between about 600 nm and about 700 nm, between about 500 nm and about 550 nm, between about 550 nm and about 600 nm, between about 600 nm and about 650 nm, or between about 650 nm and about 700 nm). In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments, a spectral range has a range of between about 5 nm and about 10 nm, between about 10 nm and about 50 nm, or between about 50 nm and about 100 nm.

### Sequencing

Aspects of the disclosure relate to sequencing biological polymers, such as polypeptides and proteins. As used herein, “sequencing,” “sequence determination,” “determining a sequence,” and like terms, in reference to a polypeptide or protein includes determination of partial sequence information as well as full sequence information of the polypeptide or protein. That is, the terminology includes sequence comparisons, fingerprinting, probabilistic fingerprinting, and like levels of information about a target molecule, as well as the express identification and ordering of each amino acid of the target molecule within a region of interest. In some embodiments, the terminology includes identifying a single amino acid of a polypeptide. In yet other embodiments, more than one amino acid of a polypeptide is identified. As used herein, in some embodiments, “identifying,” “determining the identity,” and like terms, in reference to an amino acid includes determination of an express identity of an amino acid as well as determination of a probability of an express identity of an amino acid. For example, in some embodiments, an amino acid is identified by determining a probability (e.g., from 0% to 100%) that the amino acid is of a specific type, or by determining a probability for each of a plurality of specific types. Accordingly, in some embodiments, the terms “amino acid sequence,” “polypeptide sequence,” and “protein sequence” as used herein may refer to the polypeptide or protein material itself and is not restricted to the specific sequence information (e.g., the succession of letters representing the order of amino acids from one terminus to another terminus) that biochemically characterizes a specific polypeptide or protein.

In some embodiments, methods of sequencing involve assessing the identity of a terminal amino acid. In some embodiments, the identity of a terminal amino acid (e.g., an N-terminal or a C-terminal amino acid) is assessed after which the terminal amino acid is removed and the identity of the next amino acid at the terminus is assessed, and this process is repeated until a plurality of successive amino acids in the polypeptide are assessed. In some embodiments, assessing the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

However, in some embodiments assessing the identity of a terminal amino acid type can comprise determining a subset of potential amino acids that can be present at the terminus of the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the polypeptide (e.g., using a binding agent that binds to a specified subset of two or more terminal amino acids).

In some embodiments, assessing the identity of a terminal amino acid type comprises determining that an amino acid comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination.

In some embodiments, assessing the identity of a terminal amino acid type comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and sequence information can be obtained from one or more of these smaller polypeptides (e.g., using a method that involves sequentially assessing a terminal amino acid of a polypeptide and removing that amino acid to expose the next amino acid at the terminus).

In some embodiments, a polypeptide is sequenced from its amino (N) terminus. In some embodiments, a polypeptide is sequenced from its carboxy (C) terminus. In some embodiments, a first terminus (e.g., N or C terminus) of a polypeptide is immobilized and the other terminus (e.g., the C or N terminus) is sequenced as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some embodiments, sequencing of a polypeptide molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids in the polypeptide molecule. In some embodiments, the at least two amino acids are contiguous amino acids. In some embodiments, the at least two amino acids are non-contiguous amino acids.

In some embodiments, sequencing of a polypeptide molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the polypeptide molecule. For example, in some embodiments, sequencing of a polypeptide molecule comprises identification of less than 100% of one type of amino acid in the polypeptide molecule (e.g., identification of a portion of all amino acids of one type in the polypeptide molecule). In some embodiments, sequencing of a polypeptide molecule comprises identification of less than 100% of each type of amino acid in the polypeptide molecule.

In some embodiments, sequencing of a polypeptide molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the polypeptide.

In some embodiments, the disclosure provides compositions and methods for sequencing a polypeptide by identifying a series of amino acids that are present at a terminus of a polypeptide over time (e.g., by iterative detection and cleavage of amino acids at the terminus). In yet other embodiments, the disclosure provides compositions and methods for sequencing a polypeptide by identifying labeled amino content of the polypeptide and comparing to a reference sequence database.

In some embodiments, the disclosure provides compositions and methods for sequencing a polypeptide by sequencing a plurality of fragments of the polypeptide. In some embodiments, sequencing a polypeptide comprises combining sequence information for a plurality of polypeptide fragments to identify and/or determine a sequence for the polypeptide. In some embodiments, combining sequence information may be performed by computer hardware and software. The methods described herein may allow for a set of related polypeptides, such as an entire proteome of an organism, to be sequenced. In some embodiments, a plurality of single molecule sequencing reactions are performed in parallel (e.g., on a single chip) according to aspects of the present disclosure. For example, in some embodiments, a plurality of single molecule sequencing reactions are each performed in separate sample wells on a single chip.

In some embodiments, methods provided herein may be used for the sequencing and identification of an individual protein in a sample comprising a complex mixture of proteins. In some embodiments, the disclosure provides methods of uniquely identifying an individual protein in a complex mixture of proteins. In some embodiments, an individual protein is detected in a mixed sample by determining a partial amino acid sequence of the protein. In some embodiments, the partial amino acid sequence of the protein is within a contiguous stretch of approximately 5 to 50 amino acids.

Without wishing to be bound by any particular theory, it is believed that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a complex mixture of proteins can be degraded (e.g., chemically degraded, enzymatically degraded) into short polypeptide fragments of approximately 6 to 40 amino acids, and sequencing of this polypeptide library would reveal the identity and abundance of each of the proteins present in the original complex mixture. Compositions and methods for selective amino acid labeling and identifying polypeptides by determining partial sequence information are described in detail in U.S. patent application Ser. No. 15/510,962, filed Sep. 15, 2015, titled “SINGLE MOLECULE PEPTIDE SEQUENCING,” which is incorporated herein by reference in its entirety.

Embodiments are capable of sequencing single polypeptide molecules with high accuracy, such as an accuracy of at least about 50%, 60%, 70%, 75%, 80%, 85%, 90%, 95%, 96%, 97%, 98%, 99%, 99.9%, 99.99%, 99.999%, or 99.9999%. In some embodiments, the target molecule used in single molecule sequencing is a polypeptide that is immobilized to a surface of a solid support such as a bottom surface or a sidewall surface of a sample well. The sample well also can contain any other reagents needed for a sequencing reaction in accordance with the disclosure, such as one or more suitable buffers, co-factors, labeled recognition molecules, and enzymes (e.g., catalytically active or inactive exopeptidase enzymes, which may be luminescently labeled or unlabeled).

As described above, in some embodiments, sequencing in accordance with the disclosure comprises identifying an amino acid by determining a probability that the amino acid is of a specific type. Conventional protein identification systems require identification of each amino acid in a polypeptide to identify the polypeptide. However, it is difficult to accurately identify each amino acid in a polypeptide. For example, data collected from an interaction in which a first recognition molecule associates with a first amino acid may not be sufficiently different from data collected from an interaction in which a second recognition molecule associates with a second amino acid to differentiate between the two amino acids. In some embodiments, sequencing in accordance with the disclosure avoids this problem by using a protein identification system that, unlike conventional protein identification systems, does not require (but does not preclude) identification of each amino acid in the protein.

Accordingly, in some embodiments, sequencing in accordance with the disclosure may be carried out using a protein identification system that uses machine learning techniques to identify proteins. In some embodiments, the system operates by: (1) collecting data about a polypeptide of a protein using a real-time protein sequencing device; (2) using a machine learning model and the collected data to identify probabilities that certain amino acids are part of the polypeptide at respective locations; and (3) using the identified probabilities, as a “probabilistic fingerprint” to identify the protein. In some embodiments, data about the polypeptide of the protein may be obtained using reagents that selectively bind amino acids. As an example, the reagents and/or amino acids may be labeled with luminescent labels that emit light in response to application of excitation energy. In this example, a protein sequencing device may apply excitation energy to a sample of a protein (e.g., a polypeptide) during binding interactions of reagents with amino acids in the sample. In some embodiments, one or more sensors in the sequencing device (e.g., a photodetector, an electrical sensor, and/or any other suitable type of sensor) may detect binding interactions. In turn, the data collected and/or derived from the detected light emissions may be provided to the machine learning model. Machine learning models and associated systems and methods are described in detail in U.S. Provisional Patent Appl. No. 62/860,750, filed Jun. 12, 2019, titled “MACHINE LEARNING ENABLED PROTEIN IDENTIFICATION,” which is incorporated herein by reference in its entirety.

Sequencing in accordance with the disclosure, in some aspects, may involve immobilizing a polypeptide on a surface of a substrate (e.g., of a solid support, for example a chip, for example an integrated device as described herein). In some embodiments, a polypeptide may be immobilized on a surface of a sample well (e.g., on a bottom surface of a sample well) on a substrate. In some embodiments, the N-terminal amino acid of the polypeptide is immobilized (e.g., attached to the surface). In some embodiments, the C-terminal amino acid of the polypeptide is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this disclosure. In some embodiments, a plurality of polypeptides are attached to a plurality of sample wells (e.g., with one polypeptide attached to a surface, for example a bottom surface, of each sample well), for example in an array of sample wells on a substrate.

Sequencing in accordance with the disclosure, in some aspects, may be performed using a system that permits single molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent marker, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to the integrated device and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing nucleotides or any other biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

### Disclosed Concepts

A1. A composition comprising: a first amino acid binding protein comprising a first FRET label, wherein the first FRET label has a first emission spectrum comprising peaks of a first wavelength and a second wavelength; and a second amino acid binding protein comprising a second FRET label, wherein the second FRET label has a second emission spectrum comprising peaks of the first wavelength and the second wavelength, wherein emission intensities at one or both peaks of the first emission spectrum are different from emission intensities at one or both peaks of the second emission spectrum.

A1.1. The composition of concept A1, wherein emission intensities at the first and second wavelengths in the first emission spectrum are different from emission intensities at the first and second wavelengths in the second emission spectrum.

A2. The composition of concept A1 or A1.1, wherein the first wavelength is an emission wavelength for a donor chromophore of each FRET label, and the second wavelength is an emission wavelength for an acceptor chromophore of each FRET label.

A3. The composition of concept A2, wherein the ratio of the donor chromophore to the acceptor chromophore in each FRET label is 1:1, 2:1, 3:1, 4:1, 5:1, 1:2, 1:3, 1:4, or 1:5.

A4. The composition of any one of concepts A1-A3, wherein the first FRET label has a first FRET efficiency, and the second FRET label has a second FRET efficiency, wherein the first FRET efficiency is different from the second FRET efficiency.

A5. The composition of concept A4, wherein the first FRET efficiency differs from the second FRET efficiency by at least about 5%.

A6. The composition of concept A4 or A5, wherein: the first amino acid binding protein comprises the first FRET label in a first configuration that permits the first FRET efficiency; and the second amino acid binding protein comprises the second FRET label in a second configuration that permits the second FRET efficiency.

A7. The composition of concept A6, wherein the first configuration maintains a first distance between chromophores in the first FRET label, and the second configuration maintains a second distance between the chromophores in the second FRET label, wherein the first distance is different from the second distance.

A8. The composition of concept A6 or A7, wherein the first amino acid binding protein is attached to the first FRET label through a first linkage group, and the second amino acid binding protein is attached to the second FRET label through a second linkage group.

A9. The composition of concept A8, wherein chromophores of the first FRET label are attached to the first linkage group in the first configuration, and chromophores of the second FRET label are attached to the second linkage group in the second configuration.

A10. The composition of any one of concepts A1-A9, wherein the first FRET label comprises a first chromophore, and the second FRET label comprises a second chromophore that is identical to the first chromophore.

A11. The composition of any one of concepts A1-A10, wherein the first FRET label comprises a first plurality of chromophores, the second FRET label comprises a second plurality of chromophores, and chromophores of the first plurality are identical to chromophores of the second plurality.

A12. The composition of any one of concepts A1-A11, further comprising at least one amino acid binding protein comprising a non-FRET label.

A13. The composition of concept A12, wherein the non-FRET label comprises a fluorophore.

A14. The composition of concept A12, wherein the non-FRET label comprises a chromophore identical to a donor or acceptor chromophore of the first FRET label.

A15. The composition of any one of concepts A1-A14, wherein the first emission spectrum distinctly identifies a first type of amino acid, and the second emission spectrum distinctly identifies a second type of amino acid.

A16. The composition of concept A15, wherein the first and second types of amino acids are naturally occurring amino acids of a different type.

A16.1. The composition of any one of concepts A15-A16, wherein the first and/or second types of amino acids are post-translationally modified amino acids.

A17. The composition of any one of concepts A1-A16, wherein the first amino acid binding protein binds to a first subset of types of amino acids, and the second amino acid binding protein binds to a second subset of types of amino acids.

A17.1. The composition of any one of concepts A1-A17, wherein the first amino acid binding protein distinctly identifies a first subset of types of amino acids, and the second amino acid binding protein distinctly identifies a second subset of types of amino acids.

A18. The composition of concept A17 or A17.1, wherein the first subset of types of amino acids is different from the second subset of types of amino acids.

A19. The composition of any one of concepts A1-A18, further comprising at least one peptidase.

A20. The composition of concept A19, wherein the molar ratio of the first or second amino acid binding protein to the peptidase is between about 1:1,000 and about 1:1 or between about 1:1 and about 100:1.

A21. The composition of concept A19, wherein the molar ratio of the first or second amino acid binding protein to the peptidase is between about 1:100 and about 1:1 or between about 1:1 and about 10:1.

A22. The composition of concept A19, wherein the molar ratio of the first or second amino acid binding protein to the peptidase is about 1:1,000, about 1:500, about 1:200, about 1:100, about 1:10, about 1:5, about 1:2, about 1:1, about 5:1, about 10:1, about 50:1, about 100:1.

A23. The composition of any one of concepts A1-A22, wherein the first and second amino acid binding proteins are each independently selected from a Gid protein, a UBR-box protein or UBR-box domain-containing fragment thereof, a p62 protein or ZZ domain-containing fragment thereof, and a ClpS protein.

A24. The composition of any one of concepts A1-A23, wherein at least one of the first and second amino acid binding proteins is a ClpS protein.

A25. A method of polypeptide sequencing, the method comprising: contacting a single polypeptide molecule with a composition according to any one of concepts A1-A24; and detecting a series of signal pulses indicative of association of the first and second amino acid binding proteins with the single polypeptide while the single polypeptide is being degraded, thereby sequencing the single polypeptide molecule.

B 1. A labeled amino acid recognition molecule comprising: a nucleic acid comprising a FRET label, wherein the FRET label has an emission spectrum comprising at least two peaks that distinctly identify a terminal amino acid; andat least one amino acid binding protein attached to the nucleic acid,wherein the nucleic acid forms a covalent or non-covalent linkage group between the at least one amino acid binding protein and the FRET label.

B2. The labeled amino acid recognition molecule of concept B 1, wherein the FRET label has a FRET efficiency of less than 90%.

B3. The labeled amino acid recognition molecule of concept B2, wherein the FRET label is attached to the nucleic acid in a configuration that permits the FRET efficiency.

B4. The labeled amino acid recognition molecule of any one of concepts B1-B3, wherein the FRET label comprises a plurality of chromophores attached to a respective plurality of attachment sites on the nucleic acid.

B5. The labeled amino acid recognition molecule of concept B4, wherein each attachment site is separated by another attachment site of the plurality by between 5 and 100 nucleotide bases or nucleotide base pairs on the nucleic acid.

B6. The labeled amino acid recognition molecule of any one of concepts B1-B5, wherein the FRET label is attached to the nucleic acid through a biomolecule that forms a covalent or non-covalent linkage group between the FRET label and the nucleic acid.

B7. The labeled amino acid recognition molecule of concept B6, wherein the FRET label comprises a plurality of chromophores attached to a respective plurality of attachment sites on the biomolecule.

B8. The labeled amino acid recognition molecule of concept B6 or B7, wherein the biomolecule is a multivalent protein.

B9. The labeled amino acid recognition molecule of any one of concepts B1-B8, wherein the nucleic acid is a double-stranded nucleic acid comprising a first oligonucleotide strand hybridized with a second oligonucleotide strand.

B10. The labeled amino acid recognition molecule of concept B9, wherein the at least one amino acid binding protein is attached to the first oligonucleotide strand, and wherein the FRET label is attached to the first oligonucleotide strand.

B11. The labeled amino acid recognition molecule of concept B9, wherein the at least one amino acid binding protein is attached to the first oligonucleotide strand, and wherein the FRET label is attached to the second oligonucleotide strand.

B12. The labeled amino acid recognition molecule of concept B9, wherein the at least one amino acid binding protein is attached to the first oligonucleotide strand, and wherein chromophores of the FRET label are attached to each of the first and second oligonucleotide strands.

B13. The labeled amino acid recognition molecule of any one of concepts B1-B12, wherein the FRET label comprises a donor chromophore and an acceptor chromophore, and wherein the ratio of the donor chromophore to the acceptor chromophore is 1:1, 2:1, 3:1, 4:1, 5:1, 1:2, 1:3, 1:4, or 1:5.

B14. A method of polypeptide sequencing, the method comprising: contacting a single polypeptide molecule with a composition comprising one or more amino acid recognition molecules, wherein at least one amino acid recognition molecule is a labeled amino acid recognition molecule according to any one of concepts B1-B13; and detecting a series of signal pulses indicative of association of the one or more amino acid recognition molecules with successive amino acids exposed at a terminus of the single polypeptide while the single polypeptide is being degraded, thereby sequencing the single polypeptide molecule.

C1. A composition comprising:
a first amino acid binding protein comprising a first label, wherein the first amino acid binding protein binds a first type of amino acid; and a second amino acid binding protein comprising a second label, wherein the second amino acid binding protein binds the first type of amino acid, wherein the first label is different from the second label.

C1.1 The composition of concept C1, wherein the first amino acid binding protein binds a second type of amino acid and/or the second amino acid binding protein binds the second type of amino acid.

C2. The composition of concept C1 or C1.1, wherein the first and second amino acid binding proteins are the same.

C3. The composition of concept C1 or C1.1, wherein the first and second amino acid binding proteins are different.

C4. The composition of concept C3, wherein the first amino acid binding protein binds the first type of amino acid with a first dissociation rate, and the second amino acid binding protein binds the first type of amino acid with a second dissociation rate, wherein the first dissociation rate is different from the second dissociation rate.

C5. The composition of any one of concepts C-C4, wherein the first label comprises a first fluorophore, and the second label comprises a second fluorophore, wherein the first fluorophore is different from the second fluorophore.

C6. The composition of any one of concepts C1-C5, wherein the first and second amino acid binding proteins are each independently selected from a Gid protein, a UBR-box protein or UBR-box domain-containing fragment thereof, a p62 protein or ZZ domain-containing fragment thereof, and a ClpS protein.

C7. The composition of any one of concepts C1-C6, wherein at least one of the first and second amino acid binding proteins is a ClpS protein.

C8. A method of polypeptide sequencing, the method comprising: contacting a single polypeptide molecule with a composition according to any one of concepts C1-C7; and detecting a series of signal pulses indicative of association of the first and second amino acid binding proteins with the single polypeptide while the single polypeptide is being degraded, thereby sequencing the single polypeptide molecule.

C9 A method of identifying a terminal amino acid of a polypeptide, the method comprising: contacting a single polypeptide molecule with a composition according to any one of concepts C1-C7; and detecting a series of signal pulses indicative of association of the first and second amino acid binding proteins with a terminus of the single polypeptide molecule; and identifying the first type of amino acid at the terminus of the single polypeptide molecule based on a characteristic pattern in the series of signal pulses.

C10. The method of concept C9, wherein a signal pulse of the characteristic pattern corresponds to an individual association event between the first or second amino acid binding protein and the first type of amino acid.

C11. The method of concept C10, wherein the signal pulse of the characteristic pattern comprises a pulse duration that is characteristic of a dissociation rate of binding between the first or second amino acid binding protein and the first type of amino acid.

C12. The method of concept C11, wherein association of the first amino acid binding protein with the first type of amino acid produces a first pulse duration, and association of the second amino acid binding protein with the first type of amino acid produces a second pulse duration.

C13. The method of concept C12, wherein the first pulse duration is different from the second pulse duration.

C14. The method of concept C12, wherein the first and second pulse durations are the same.

D1. A system comprising: at least one hardware processor; and at least one non-transitory computer-readable storage medium storing processor-executable instructions that, when executed by the at least one hardware processor, cause the at least one hardware processor to perform the method of any of concepts A25, B14, or C8-C14.

D2. At least one non-transitory computer-readable storage medium storing processor-executable instructions that, when executed by at least one hardware processor, cause the at least one hardware processor to perform the method of any of concepts A25, B14, or C8-C14.

E1. An integrated device comprising: at least one chamber for receiving one or more labeled amino acid binding proteins; at least one photodetection region for receiving a signal emitted by the one or more labeled amino acid binding proteins in response to excitation light from at least one light source, the signal including information representative of at least one characteristic of the one or more labeled amino acid binding proteins; and at least one controller configured to obtain one or more adjusted measurements by controlling adjusting of one or more subsequent measurements obtained from a single polypeptide molecule disposed in the at least one chamber based on the information obtained from the signal emitted by the one or more labeled amino acid binding proteins.

E2. The integrated device of concept E1, wherein the one or more labeled amino acid binding proteins comprise at least one amino acid binding protein comprising a FRET label, wherein the FRET label has an emission spectrum comprising peaks of a first wavelength and a second wavelength.

E3. The integrated device of concept E1, wherein the one or more labeled amino acid binding proteins comprise: a first amino acid binding protein comprising a first FRET label, wherein the first FRET label has a first emission spectrum comprising peaks of a first wavelength and a second wavelength; and a second amino acid binding protein comprising a second FRET label, wherein the second FRET label has a second emission spectrum comprising peaks of the first wavelength and the second wavelength, wherein emission intensities at the first and second wavelengths in the first emission spectrum are different from emission intensities at the first and second wavelengths in the second emission spectrum.

E4. The integrated device of concept E1, wherein the one or more labeled amino acid binding proteins comprise: a first amino acid binding protein comprising a first label, wherein the first amino acid binding protein binds a first type of amino acid; and a second amino acid binding protein comprising a second label, wherein the second amino acid binding protein binds the first type of amino acid, wherein the first label is different from the second label.

E5. The integrated device of concept E1, wherein the at least one characteristic of the labeled amino acid binding protein comprises a luminescence intensity, a luminescence wavelength, a luminescence lifetime, a pulse duration, and/or an interpulse duration.

E6. The integrated device of concept E1, wherein the one or more adjusted measurements are representative of a luminescence intensity, a luminescence wavelength, a luminescence lifetime, a pulse duration, and/or an interpulse duration.

E7. The integrated device of concept E1, wherein the at least one controller is configured to identify one or more amino acids of the single polypeptide molecule based at least in part on the one or more adjusted measurements.

E8. The integrated device of concept E1, wherein the at least one controller is configured to identify the single polypeptide molecule, or a protein from which the single polypeptide molecule is derived, at least in part by identifying one or more amino acids of the single polypeptide molecule based at least in part on the one or more adjusted measurements.

E9. The integrated device of concept E1, wherein: the at least one chamber comprises a plurality of chambers having a respective plurality of single polypeptide molecules disposed therein; the one or more labeled amino acid binding proteins comprise a plurality of labeled amino acid binding proteins; the at least one photodetection region comprises a plurality of photodetection regions configured to receive signals from the plurality of labeled amino acid binding proteins; and the at least one controller is configured to control the adjusting of the one or more subsequent measurements obtained respectively from each of the plurality of single polypeptide molecules based on information obtained from the plurality of signals emitted by the plurality of labeled amino acid binding proteins.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A composition comprising:
a first amino acid binding protein comprising a first FRET label, wherein the first FRET label has a first emission spectrum comprising peaks of a first wavelength and a second wavelength; and
a second amino acid binding protein comprising a second FRET label, wherein the second FRET label has a second emission spectrum comprising peaks of the first wavelength and the second wavelength,
wherein emission intensities at one or both peaks of the first emission spectrum are different from emission intensities at one or both peaks of the second emission spectrum.

2. The composition of claim 1, wherein the first wavelength is an emission wavelength for a donor chromophore of each FRET label, and the second wavelength is an emission wavelength for an acceptor chromophore of each FRET label.

3. The composition of claim 2, wherein the ratio of the donor chromophore to the acceptor chromophore in each FRET label is 1:1, 2:1, 3:1, 4:1, 5:1, 1:2, 1:3, 1:4, or 1:5.

4. The composition of claim 1, wherein the first FRET label has a first FRET efficiency, and the second FRET label has a second FRET efficiency, wherein the first FRET efficiency is different from the second FRET efficiency.

5. The composition of claim 4, wherein the first FRET efficiency differs from the second FRET efficiency by at least about 5%.

6. The composition of claim 5, wherein:
the first amino acid binding protein comprises the first FRET label in a first configuration that permits the first FRET efficiency; and
the second amino acid binding protein comprises the second FRET label in a second configuration that permits the second FRET efficiency.

7. The composition of claim 6, wherein the first configuration maintains a first distance between chromophores in the first FRET label, and the second configuration maintains a second distance between chromophores in the second FRET label, wherein the first distance is different from the second distance.

8. The composition of claim 1, wherein the first amino acid binding protein is attached to the first FRET label through a first linkage group, and the second amino acid binding protein is attached to the second FRET label through a second linkage group.

9. The composition of claim 8, wherein chromophores of the first FRET label are attached to the first linkage group in the first configuration, and chromophores of the second FRET label are attached to the second linkage group in the second configuration.

10. The composition of claim 1, wherein the first FRET label comprises a first chromophore, and the second FRET label comprises a second chromophore that is identical to the first chromophore.

11. The composition of claim 1, wherein the first FRET label comprises a first plurality of chromophores, the second FRET label comprises a second plurality of chromophores, and chromophores of the first plurality are identical to chromophores of the second plurality.

12. The composition of claim 1, further comprising at least one amino acid binding protein comprising a non-FRET label.

13. The composition of claim 12, wherein the non-FRET label comprises a fluorophore.

14. The composition of claim 12, wherein the non-FRET label comprises a chromophore identical to a donor or acceptor chromophore of the first FRET label.

15. The composition of claim 1, wherein the first emission spectrum distinctly identifies a first type of amino acid, and the second emission spectrum distinctly identifies a second type of amino acid.

16. The composition of claim 15, wherein the first and second types of amino acids are naturally occurring amino acids of a different type.

17. The composition of claim 1, wherein the first amino acid binding protein binds to a first subset of types of amino acids, and the second amino acid binding protein binds to a second subset of types of amino acids.

18. The composition of claim 1, further comprising at least one peptidase.

19. A labeled amino acid recognition molecule comprising:
a nucleic acid comprising a FRET label, wherein the FRET label has an emission spectrum comprising at least two peaks that distinctly identify a terminal amino acid; and
at least one amino acid binding protein attached to the nucleic acid,
wherein the nucleic acid forms a covalent or non-covalent linkage group between the at least one amino acid binding protein and the FRET label.

20. A composition comprising:
a first amino acid binding protein comprising a first label, wherein the first amino acid binding protein binds a first type of amino acid; and
a second amino acid binding protein comprising a second label, wherein the second amino acid binding protein binds the first type of amino acid,
wherein the first label is different from the second label.


SEQUENCE LISTING (8 sequences)

SEQ ID NO: 1 - protein, 27 aa, Synthetic construct
GGGSGGGSGGGSGLNDFFEAQKIEWHE

SEQ ID NO: 2 - protein, 54 aa, Synthetic construct
GGGSGGGSGGGSGLNDFFEAQKIEWHEGGGSGGGSGGGSGLNDFFEAQKIEWHE

SEQ ID NO: 3 - protein, 56 aa, Synthetic construct
GSGGGSGGGSGGGSGLNDFFEAQKIEWHEGGGSGGGSGGGSGLNDFFEAQKIEWHE

SEQ ID NO: 4 - protein, 38 aa, Synthetic construct
GHHHHHHHHHHGGGSGGGSGGGSGLNDFFEAQKIEWHE

SEQ ID NO: 5 - protein, 65 aa, Synthetic construct
GHHHHHHHHHHGGGSGGGSGGGSGLNDFFEAQKIEWHEGGGSGGGSGGGSGLNDFFEAQKIEWHE

SEQ ID NO: 6 - protein, 67 aa, Synthetic construct
GGSHHHHHHHHHHGGGSGGGSGGGSGLNDFFEAQKIEWHEGGGSGGGSGGGSGLNDFFEAQKIEWHE

SEQ ID NO: 7 - protein, 66 aa, Synthetic construct
GSHHHHHHHHHHGGGSGGGSGGGSGLNDFFEAQKIEWHEGGGSGGGSGGGSGLNDFFEAQKIEWHE

SEQ ID NO: 8 - protein, 61 aa, Synthetic construct
GGGSGGGSGGGSGLNDFFEAQKIEWHEGGGSGGGSGGGSGLNDFFEAQKIEWHEGHHHHHH